(19) United States
(12) Patent Application Publication
(10) Pub. No.: US 2005/0282261 A1
(43) Pub. Date: Dec. 22, 2005

(54) Novel choline oxidases

(75) Inventors: Kerstin Sauter, Dormagen-Nievenheim (DE); Albrecht Weiss, Langenfeld (DE); Karl-Heinz Maurer, Erkrath (DE); Stefan Evers, Mettmann (DE); Nina Hoven, Moenchengladbach (DE); Susanne Wieland, Dormagen-Zons (DE); Regina Stehr, Neuss (DE); Cornelius Bessler, Duesseldorf (DE)

Correspondence Address: WOODCOCK WASHBURN LLP, ONE LIBERTY PLACE, 46TH FLOOR, PHILADELPHIA, PA, 19103, US

(73) Assignee: Henkel Kommanditgesellschaft auf Aktien, Duesseldorf (DE)

(21) Appl. No.: 11/156,003

(22) Filed: Jun. 17, 2005

Related U.S. Application Data
(63) Continuation of application No. PCT/EP03/14130, filed on Dec. 12, 2003.

(30) Foreign Application Priority Data
Dec. 20, 2002 (DE) DE 102 60 930.6

(51) Int. Cl. C12N 9/00

(52) U.S. Cl. 435/183

(57) ABSTRACT

The invention relates to novel choline oxidases, methods for obtaining the same, bodycare products, haircare products, shampoos, oral, dental and dental prosthesis care products, cosmetics, washing products, cleaning products, rinsing products, hand soaps, washing-up detergents and dishwasher detergents comprising novel choline oxidases and uses of the novel choline oxidases.

### CROSS REFERENCE TO RELATED APPLICATIONS

This application is a continuation of PCT/EP2003/014130, filed Dec. 12, 2003, which claims priority to DE 102 60 930.6, filed Dec. 12, 2002, the disclosures of which are incorporated herein in their entireties.

### DETAILED DESCRIPTION OF ILLUSTRATIVE EMBODIMENTS

The present invention relates to novel choline oxidases, to processes for isolating them, to bodycare products, hair care products, shampoos, oral care, tooth care and denture care products, cosmetics, detergents, cleaning agents, rinsing agents, hand washing detergents, hand dishwashing detergents and machine dishwashing detergents containing novel choline oxidases and to uses of the novel choline oxidases.

Choline oxidases are known from the prior art (Ikuta, S., Imamura, S., Misaki, H., and Horiuti, Y. 1977. Purification and characterization of choline oxidase from Arthrobacter globiformis. J Biochem (Tokyo) 82: 1741-1749., Deshnium, P., Los, D. A., Hayashi, H., Mustardy, L., and Murata, N. 1995. Transformation of Synechococcus with a gene for choline oxidase enhances tolerance to salt stress. Plant Mol Biol 29: 897-907). It is also known that oxidases (alcohol oxidases, amino acid oxidases) may be used together with their substrates for generating hydrogen peroxide for bleaching and dye transfer inhibition in detergents or for enzymatic hair dyeing and bleaching in cosmetic preparations.

WO 97/21796 describes that oxidases release hydrogen peroxide from their corresponding substrates under industrial conditions (for example a detergent matrix) with the assistance of atmospheric oxygen and may accordingly be used for bleaching. Hydrogen peroxide formation proceeds continuously, the efficiency of product formation being determined by temperature stability and pH stability, and tolerance towards substrate and product.

In conventional chemical bleaching, inorganic, alkaline hydrogen peroxide sources such as percarbonate or perborate are used in combination with bleach boosters (TAED). The bleaching component hydrogen peroxide arises by spontaneous decomposition of the addition compound and so rapidly but transiently gives rise to elevated concentrations. Gentle bleaching is not possible.

There is no ideal bleaching system available for use in aqueous liquid formulations.

In hair dyeing, bleaching with hydrogen peroxide and ammonia solution is conventionally performed to even out gray shades prior to dyeing. The transient high hydrogen peroxide concentrations in combination with the alkaline pH cause appreciable hair damage. There is no pretreatment available, which does not use ammonia, which causes an odor nuisance, combined with continuous release of hydrogen peroxide at a neutral or weakly basic pH.

It has surprisingly been found that choline oxidases may be isolated from the bacteria Arthrobacter nicotianae and Arthrobacter aurescens which may be used in bleaching systems while largely avoiding the disadvantages associated with the relevant prior art.

The subject of the present invention is accordingly a choline oxidase, the amino acid sequence of which matches the amino acid sequence stated in Seq. 1 to at least 62.5%, at least 70%, at least 75%, at least 80%, preferably at least 85%, in particular at least 90%, particularly preferably at least 95% and very particularly preferably 100%.

The present invention further provides a choline oxidase, the amino acid sequence of which matches the amino acid sequence stated in Seq. 2 to at least 72.4%, at least 75%, at least 80%, preferably at least 85%, in particular at least 90%, particularly preferably at least 95% and very particularly preferably 100%.

The present invention further provides a choline oxidase, the amino acid sequence of which matches the amino acid sequence stated in Seq. 3 to at least 68.9%, at least 70%, at least 75%, at least 80%, preferably at least 85%, in particular at least 90%, particularly preferably at least 95% and very particularly preferably 100%.

The present invention further provides a choline oxidase obtainable by single or multiple conservative amino acid exchange from a choline oxidase according to the invention or by derivatization, fragmentation, deletion mutation or insertion mutation of a choline oxidase according to the invention.

The choline oxidases according to the invention are based on novel alkaline-oxidases isolated for the first time by the applicant from Arthrobacter nicotianae (Seq. 1), Arthobacter aurescens (Seq. 2) and on a hybrid choline oxidase fused from portions of the two above-stated enzymes (Seq. 3).

The choline oxidases according to the invention are capable of continuously releasing hydrogen peroxide from choline and choline derivatives with the assistance of atmospheric oxygen with formation of betaine aldehyde and betaine.

The choline oxidases according to the invention advantageously exhibit an elevated specific rate of hydrogen formation.

The pH profile of the enzymes according to the invention is compatible with the necessary pH for industrial use and with typical products such as detergents and cleaning agents and hair dyes.

Use of the substrate choline and the derivatives thereof together with the formation of the secondary product betaine as a potential finishing active substance, gives rise to considerable secondary benefits.

Substrates, which may, for example, be considered, are choline and derivatives of N-substituted aminoethanol, with the structural formulae 1 or 2:

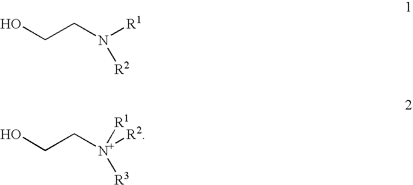

The following definitions apply to structural formula 1:
R₁=H, R²=2-hydroxyethyl
R₁=methyl, R²=methyl
R₁=2-hydroxyethyl, R²=2-hydroxyethyl

These compounds are known from the literature as substrates for the choline oxidase from A. globiformis.

The following definitions apply to structural formula 2:
R¹=R²=R³=methyl

Another substrate which is suitable according to the invention is betaine aldehyde (OHC—CH₂)N⁺(CH₃)₃

The additional hair care action of the betaine, which forms, is also of considerable interest.

Hereafter, the expression of the form “at least X %” means “X % to 100% (including the limit values X and 100 and all integral and nonintegral percentages there between)”.

For the purposes of the present application, a protein should be taken to mean a largely linear polymer made up of natural amino acids and usually assumes a three-dimensional structure in order to perform its function. In the present application, the 19 proteinogenic, naturally occurring L-amino acids are referred to by the conventional international 1 and 3 letter codes.

For the purposes of the present application, an enzyme should be taken to mean a protein, which performs a specific biocatalytic function.

Numerous proteins are formed as “preproteins”, i.e. together with a signal peptide. This should then be taken to mean the N-terminal end of the protein, the function of which is generally to ensure transfer of the protein formed out of the producing cell into the periplasm or surrounding medium and/or the correct folding thereof. Under natural conditions, the signal peptide is then cleaved from the remainder of the protein by a signal peptidase, such that the protein performs its actual catalytic activity without the initially present N-terminal amino acids.

In industrial applications, because of their enzymatic activity, it is the mature peptides, i.e. the enzymes processed after their production, which are preferred over preproteins.

Proproteins are inactive protein precursors. Their precursors with a signal sequence are described as preproproteins.

For the purposes of the present application, nucleic acids should be taken to mean the molecules built up from nucleotides, which serve as information storage media and code for the linear amino acid sequence in proteins or enzymes. They may present the form of a single strand, a single strand complementary to said single strand or a double strand. Being the naturally more durable information storage medium, the nucleic acid DNA is preferred for molecular biological work. On the other hand, RNA is formed to carry out the invention in a natural environment, such as for example in an expressing cell, for which reason RNA molecules essential to the invention likewise constitute embodiments of the present invention.

In the case of DNA, the sequences of the two complementary strands should in each case be considered in all three possible reading frames. It should furthermore be borne in mind that different codon triplets may code for the same amino acids, such that a specific amino acid sequence may be derived from two or more different nucleotide sequences which possibly exhibit only slight identity (degeneracy of the genetic code). Moreover, different organisms exhibit differences in their use of these codons. For these reasons, it is necessary to include both amino acid sequences and nucleotide sequences within the scope of protection and any stated nucleotide sequences should in each case be viewed merely as an exemplary coding for a specific succession of amino acids.

For the purposes of the present application, the information unit corresponding to a protein is described as a gene.

The present invention comprises the production of recombinant proteins. According to the invention, this should be taken to include any genetic engineering or microbiological processes based on introducing the genes for the proteins in question into a host organism suitable for production and having them transcribed and translated by said organism. The genes in question are suitably introduced via vectors, in particular expression vectors; but also those which ensure that the gene in question may be inserted in the host organism into a preexisting genetic element such as the chromosome or other vectors. The functional unit comprising gene and promoter and possible further genetic elements is described according to the invention as an expression cassette. It need not necessarily be present as a physical unit for this purpose.

Using methods which are today generally known, such as for example chemical synthesis or the polymerase chain reaction (PCR) in conjunction with standard methods of molecular biology and/or protein chemistry, it is possible for the person skilled in the art, with reference to known DNA and/or amino acid sequences, to produce the corresponding nucleic acids or even complete genes. Such methods are known, for example, from Sambrook, J., Fritsch, E. F. and Maniatis, T. 2001. Molecular cloning: a laboratory manual, 3rd edition Cold Spring Laboratory Press.

Modifications to the nucleotide sequence, such as may for example be brought about by per se known molecular biological methods, are known as mutations. Depending on the type of modification, these may be described as deletion, insertion or substitution mutations or those in which different genes or parts of genes are fused or recombined with one another; these are gene mutations. The associated organisms are described as mutants. The proteins derived from mutated nucleic acids are described as variants. Accordingly, deletion, insertion or substitution mutations or fusions for example give rise to deletion, insertion or substitution mutated genes or fusion genes and, at the protein level, respectively to corresponding deletion, insertion or substitution variants or fusion proteins.

Fragments are taken to mean any proteins or peptides, which are smaller than natural proteins or those, which correspond to fully translated genes and may, for example, also be obtained by synthesis. On the basis of their amino acid sequences, they may be assigned to the relevant complete proteins. They may, for example, assume identical structures or exert proteolytic activities or sub activities, such as for example complexation of a substrate. Fragments and deletion variants of starting proteins are in principle of the same nature; while fragments tend to be smaller pieces, deletion mutants tend to lack only short domains, and thus only individual sub functions.

At the nucleic acid level, the sub-sequences correspond to the fragments.

For the purposes of the present application, chimeric or hybrid proteins should be taken to mean those which are coded by nucleic acid chains, which originate naturally from different organisms or the same organism. This procedure is also known as recombination mutagenesis. The purpose of such recombination may, for example, be to bring about or modify a specific enzymatic function by means of the protein moiety fused thereto. For the purposes of the present invention, it is immaterial whether such a chimeric protein consists of an individual polypeptide chain or two or more subunits, among which different functions may be distributed.

Proteins obtained by insertion mutation should be taken to mean those variants, which have been obtained by per se known methods by insertion of a nucleic acid or protein fragment into the starting sequences. Due to their fundamentally identical nature, they should be classed among chimeric proteins. They differ from these solely in terms of the size of the unmodified protein moiety relative to the size of the entire protein. In such insertion-mutated proteins, the proportion of foreign protein is smaller than in chimeric proteins.

Inversion mutagenesis, namely a partial inversion of the sequence, may be viewed as a special form both of deletion and of insertion. The same also applies to a new grouping of different molecular moieties, which deviates from the original amino acid sequence. It may be viewed not only as a deletion variant, but also as an insertion variant and as a shuffling variant of the original protein.

For the purposes of the present application, derivatives are taken to mean such proteins, the pure amino acid chain of which has been chemically modified. Such derivatization may proceed, for example, biologically in connection with protein biosynthesis by the host organism. Molecular biological methods may be used for this purpose. Derivatization may, however, also be performed chemically, for instance by the chemical transformation of an amino acid side chain or by covalent bonding of another compound onto the protein. Such a compound may, for example, also comprise other proteins, which are bound to the proteins according to the invention for example via difunctional chemical compounds. Such modifications may, for example, influence substrate specificity or the strength of binding to the substrate or bring about temporary blocking of enzymatic activity, if the coupled substance is an inhibitor. This may, for example, be appropriate for the period of storage. Derivatization should likewise be taken to mean covalent binding to a macromolecular support.

Proteins may, however, also be combined into groups of immunologically related proteins by reaction with an antiserum or a specific antibody. The members of a group are characterized in that they comprise the same antigenic determinant recognized by an antibody.

For the purposes of the present invention, all enzymes, proteins, fragments and derivatives, unless they need to be explicitly referred to as such, are denoted by the superordinate term proteins.

For the purposes of the present invention, vectors are taken to mean elements consisting of nucleic acids, which contain a gene of interest as the characterizing nucleic acid domain. They are capable of establishing said gene as a stable genetic element in a species or cell line over two or more generations or cell divisions. In particular when bacteria are used, vectors are specific plasmids, i.e. circular genetic elements. In genetic engineering, a distinction is drawn on the one hand between those vectors used in storage and to a certain extent also in genetic engineering work, which are known as “cloning vectors”, and, on the other hand, those which perform the function of producing the gene in question in the host cell, i.e. which enable expression of the relevant protein. These vectors are known as expression vectors.

By making a comparison with known enzymes, which are for example deposited in publicly accessible databases, it is possible to draw conclusions about the enzymatic activity of an enzyme under consideration on the basis of its amino acid or nucleotide sequence. This activity may be qualitatively or quantitatively modified by other domains of the protein, which are not involved in the actual reaction. This may, for example, relate to enzyme stability, activity, reaction conditions or substrate specificity.

Such a comparison is made by assigning similar sequences in the nucleotide or amino acid sequences of the proteins under consideration to one another. This is known as homology analysis. A tabular arrangement of the relevant positions is known as an alignment. When analyzing nucleotide sequences, both complementary strands and in each case all three possible reading frames must again be taken into consideration, as must the degeneracy of the genetic code and organism-specific codon usage. Alignments can now be produced using computer software, such as for example by the FASTA or BLAST algorithms; this procedure is described, for example, by D. J. Lipman and W. R. Pearson (1985) in Science, volume 227, pp. 1435-1441.

A compilation of all the matching positions in the compared sequences is known as a consensus sequence.

Such a comparison also allows a statement to be made regarding the similarity or homology of the compared sequences. This is stated as percentage identity, i.e. the proportion of identical nucleotides or amino acid residues in the same positions. A looser definition of homology includes conserved amino acid exchanges in this value. This is then known as percentage similarity. Such statements may be made with regard to whole proteins or genes or only to individual domains.

Producing an alignment is the first step to defining a sequence space. This hypothetical space comprises all the sequences derivable by permutation in individual positions, these sequences being obtained by taking account of all the variations occurring in the relevant individual positions of the alignment. Each hypothetically possible protein molecule forms a point in this sequence space. For example, two amino acid sequences, which, while largely identical, comprise in each case two different amino acids in only two locations, establish a sequence space of four different amino acid sequences. A very large sequence space is obtained if further homologous sequences are in each case found for individual sequences of a space. Such high levels of homology, which arise in each case in pairs, may mean that sequences with even a very low level of homology are recognized as belonging to a sequence space.

Homologous domains of different proteins are defined by matches in the amino acid sequence. These may also be characterized by an identical function. This extends to complete identity in minuscule domains or “boxes” which comprise only a few amino acids and usually perform functions essential to overall activity. The functions of the homologous domains should be taken to mean minuscule sub functions of the function performed by the entire protein, such as for example the formation of individual hydrogen bonding bonds for complexing a substrate or transition complex.

In the context of the present invention, the nucleic acid is suitably cloned into a vector. The molecular biological dimension of the invention accordingly comprises vectors with the genes for the corresponding proteins. These may, for example, include those derived from bacterial plasmids, from viruses or from bacteriophages, or predominantly synthetic vectors or plasmids with elements of the most varied origin. With the further genetic elements present in each case, vectors are capable of establishing themselves as stable units in the host cells in question over two or more generations. It is immaterial for the purposes of the invention whether they are established extrachromasomally as independent units or incorporated into a chromosome. Which particular system is selected from among the numerous systems known from the prior art is determined on a case-by-case basis. Decisive factors may be, for example, the achievable copy number, the available selection systems, most especially antibiotic resistance, or the culturability of the host cells capable of accepting the vector.

Vectors are suitable starting points for molecular biological and biochemical investigations of the gene in question or associated protein and for further developments according to the invention and ultimately for the amplification and production of proteins according to the invention. They constitute embodiments of the present invention to the extent that the sequences of the contained nucleic acid domains according to the invention in each case fall within the homology ranges described in greater detail above.

Preferred embodiments of the present invention are cloning vectors. These are suitable, apart from for storage, biological amplification or selection of the relevant gene, for characterizing the gene in question, for instance by drawing a restriction map or sequencing. Cloning vectors are also preferred embodiments of the present invention, because they constitute a transportable and storable form of the claimed DNA. They are also preferred starting points for molecular biological techniques not associated with cells, such as for example the polymerase chain reaction.

Expression vectors are chemically similar to the cloning vectors, but differ in those subsequences, which enable them to replicate in host organisms, optimized for the production of proteins and to cause the contained gene to be expressed there. Preferred embodiments are expression vectors which themselves bear the genetic elements necessary for expression. Expression is, for example, influenced by promoters, which control transcription of the gene. Expression may thus proceed not only by the natural promoter originally located upstream from this gene, but also by a host cell promoter provided by genetic engineering fusion and by a promoter provided on the expression vector as well as by a modified promoter or a completely different promoter from another organism.

Preferred embodiments are those expression vectors, which are controllable by modifying culture conditions or adding specific compounds, such as for example cell density or specific factors. Expression vectors make it possible to produce the associated protein heterologously, i.e. in an organism other than the one from which it may naturally be isolated. Homologous protein isolation from a host organism, which naturally expresses the gene by means of a suitable vector, also falls within the scope of protection of the present invention. This may have the advantage that natural modification reactions associated with translation may be performed on the resultant protein in exactly the same manner as they would proceed naturally.

Embodiments of the present invention may also comprise cell-free expression systems in which protein biosynthesis is carried out in vitro. Such expression systems are likewise established in the prior art.

In vivo synthesis of an enzyme according to the invention, i.e. by living cells, entails transferring the associated gene into a host cell, which is known as transformation. All organisms, i.e. prokaryotes or eukaryotes, are in principle suitable as host cells. Preferred host cells are those, which may be readily handled genetically, for example with regard to transformation with the expression vector and the stable establishment thereof, for example unicellular fungi or bacteria. Preferred host cells are additionally characterized by good microbiological and biotechnological handling properties. This relates, for example, to easy culturability, high growth rates, modest requirements with regard to fermentation media and good production and secretion rates for foreign proteins. The optimum expression systems for individual cases must often be identified experimentally from the wide range of systems available according to the prior art. In this manner, each protein according to the invention may be obtained from a plurality of host organisms.

Preferred embodiments comprise those host cells whose activity may be controlled thanks to genetic control elements, which are provided for example on the expression vector, or alternatively may already be present in these cells from the outset. For example, expression from these cells may be stimulated by controlled addition of chemical compounds serving as activators, by modification of culture conditions or once a specific cell density has been achieved. This enables highly economic production of the proteins in question.

Preferred host cells are prokaryotic or bacterial cells. Bacteria are differentiated from eukaryotes by generally having shorter generation times and being less demanding with regard to culture conditions. As a result, it is possible to set up cost-effective processes for isolating proteins according to the invention. In the case of gram-negative bacteria, such as for example Escherichia coli (E. coli), many proteins are secreted into the periplasmatic space, i.e. into the compartment between the two membranes enclosing the cells. This may be advantageous for specific applications. In contrast, gram-positive bacteria, such as for example bacilli or actinomycetes or other representatives of the Actinomycetales, do not have an outer membrane, which means that secreted proteins are immediately released into the nutrient medium surrounding the cells, from which, according to another preferred embodiment, the expressed proteins according to the invention may be directly purified.

One variant of this test principle are expression systems in which additional genes, for example those provided on other vectors, influence the production of proteins according to the invention. These may comprise modifying gene products or such products, which are to be purified together with the protein according to the invention, for, instance in order to influence its enzymatic function. These may be, for example, other proteins or enzymes, inhibitors or those elements, which influence interaction with different substrates.

Due to the extensive experience, for example in terms of microbiological methods and culturability, which has been gained with coliform bacteria, these bacteria comprise preferred embodiments of the present invention. Particularly preferred are those from the genera Escherichia coli, in particular non-pathogenic strains suitable for biotechnological production.

Representative members of these genera are the K12 derivatives and B strains of Escherichia coli. Strains, which may be derived from these using per se known genetic and/or microbiological methods, and may accordingly be viewed as the derivatives thereof, are of the greatest significance for genetic and microbiological work and are preferably used for the development of processes according to the invention. Such derivatives may modified, for example by deletion or insertion mutagenesis, with regard to their requirements for culture conditions, may comprise other or additional selection markers or express other or additional proteins. They may in particular comprise those derivatives, which, in addition to the protein produced according to the invention, express further economically valuable proteins.

Further preferred microorganisms are those characterized by having been obtained after transformation with one of the above-described vectors. These may, for example, comprise cloning vectors, which have been introduced into any desired bacterial strain for storage and/or modification. Such steps are widespread in the storage and further development of relevant genetic elements. Since the relevant genetic elements may be transferred from these microorganisms directly into gram-negative bacteria suitable for expression, the above transformation products also comprise embodiments of the subject matter of the invention.

Eukaryotic cells may also be suitable for the production of proteins according to the invention. Examples of these are fungi such as actinomycetes or yeasts such as Saccharomyces or Kluyveromyces. This may, for example, be particularly advantageous if the proteins are to undergo specific modifications in connection with their synthesis, which are enabled by such systems. Examples of such modifications include binding of low molecular weight compounds such as membrane anchors or oligosaccharides.

The host cells of the process according to the invention are cultured and fermented in a manner known per se, for example in batch or continuous systems. In the first case, a suitable nutrient medium is inoculated with the organism and, after a period, which must be determined experimentally, the product is harvested from the medium. Continuous fermentation operations are distinguished by the establishment of a dynamic equilibrium in which, over a comparatively long period, some cells die but are also replaced by new ones and product may simultaneously be removed from the medium.

Fermentation processes are well known per se from the prior art and constitute the actual full industrial scale production step, which is followed by a suitable purification method.

All fermentation processes based on one of the above-explained processes for the production of recombinant proteins are accordingly preferred embodiments of the subject matter of the invention.

Optimum conditions for the production processes used, for the host cells and/or the proteins to be produced must here in each case be determined experimentally with reference to the previously optimized culture conditions of the strains in question using the knowledge of a person skilled in the art, for example with regard to fermentation volume, medium composition, oxygen supply or stirrer speed.

Fermentation processes, which are characterized by the fermentation being performed via a feed strategy, may also be considered. This involves replenishing those constituents of the medium, which are consumed by the ongoing culturing; a replenishment strategy is another name for this approach. In this manner, it is possible to achieve considerable increases, not only in cell density, but also in biomass solids and/or above all in the activity of the relevant protein.

Analogously, the fermentation may also be arranged such that unwanted metabolic products are filtered out or neutralized by the addition of buffer or individually suitable counterions.

The protein produced may subsequently be harvested from the fermentation medium. This fermentation process is preferred over working up the product from the dry solids, but requires the provision of suitable secretion markers and transport systems.

In the absence of secretion, purification of the protein from the cell mass may be necessary under certain circumstance and various processes are also known for this purpose, such as precipitation, for example by ammonium sulfate or ethanol, or chromatographic purification, if necessary continued to homogeneity. For the majority of the described industrial processes, however, an enriched, stabilized preparation should be adequate.

All the elements already explained above may be combined to form a process to produce proteins according to the invention. A plurality of possible combinations of process steps is here conceivable for each protein according to the invention. The optimum process must be determined experimentally for each specific individual case.

The choline oxidases according to the invention may be provided in the quantity required for industrial use by expression or cloning.

The present invention accordingly further provides a nucleic acid which codes for a choline oxidase, the amino acid sequence of which contains a portion which is at least 60%, at least 70%, preferably at least 80%, in particular at least 90%, particularly preferably at least 95% and very particularly preferably 100% identical to one of the amino acid sequences stated in Seq. 1 to 3.

The present invention further provides a nucleic acid which codes for choline oxidase, the nucleotide sequence of which nucleic acid at least 78%, at least 80%, preferably at least 85%, in particular at least 90%, particularly preferably at least 95% and very particularly preferably 100% matches the nucleotide sequence stated in Seq. 4.

The present invention further provides a nucleic acid, which codes for choline oxidase, the nucleotide sequence of which nucleic acid at least 83.3%, at least 85%, particularly preferably at least 90%, and very particularly preferably 100% matches the nucleotide sequence stated in Seq. 5.

The present invention further provides a nucleic acid which codes for choline oxidase, the nucleotide sequence of which nucleic acid at least 81.5%, at least 85%, in particular at least 90%, particularly preferably at least 95% and very particularly preferably 100% matches the nucleotide sequence stated in Seq. 6.

The choline oxidases according to the invention exhibit an optimum pH preferably in the almost neutral to weakly alkaline range of around pH 6 to pH 10, particularly preferably pH 7 to pH 9. The activity of such enzymes is conventionally stated in U, the unit corresponding to that quantity of enzyme that generates 1 μmol of hydrogen peroxide (H₂O₂) at a specified pH and a specified temperature in 1 minute. For the choline oxidases according to the invention, this refers to a pH of 9.5 and a temperature of 30° C. in the method stated in Example 6.

The optimum temperature of the choline oxidases according to the invention is for instance in the range from 20 to 60° C., in particular at around 30° C.

A choline oxidase according to the invention is preferably used in quantities such that complete agent exhibits oxidase activity of 3 U/g to 20,000 U/g, preferably of 5 U/g to 20,000 U/g, in particular of 10 U/g to 15,000 U/g, particularly preferably of 10 U/g to 1000 U/g, very particularly preferably of 10 to 200 U/g. Agents with oxidase activities in the stated ranges exhibit sufficiently rapid hydrogen peroxide release for conventional European machine washing cycles, while increasing the quantity of oxidase present to higher levels of activity does not generally give rise to a correspondingly large increase in bleaching performance.

The quantity of oxidase substrate present in the detergent according to the invention is determined on the basis of the quantity of hydrogen peroxide required to achieve the desired bleaching result. By way of guidance, enzyme-substrate systems release up to two moles of hydrogen peroxide per mole of converted substrate. The presence of around 0.05 wt. % to 1 wt. % of substrate in the washing, bleaching or cleaning liquor is generally sufficient to achieve a good bleaching result.

Homologies of the choline oxidases according to the invention were determined by means of a BLAST search in public domain databases (GenBank at NCBI): search criteria

Database: All non-redundant GenBank CDStranslations+PDB+SwissProt+PIR+PRF
Search query:
COD_{—}Arthrobacter_{—}nicotianae_KC2
COD_{—}Arthrobacter_aurescens
COD_Hybrid-Protein

In every case, the best hit was choline oxidase from Arthrobacter globiformis (see Table 1).

The database references for choline oxidase from Arthrobacter globiformis read: pir∥S52489 choline oxidase (EC 1.1.3.17)—Arthrobacter globiformis pir∥S62689 choline oxidase (EC 1.1.3.17)—Arthrobacter globiformis emb|CAA59321.1|(X84895) choline oxidase.

Amino acid identity values for the whole choline oxidase protein from Arthrobacter globiformis amount to
COD_{—}Arthrobacter_{—}nicotianae_KC2=62.4%
COD_{—}Arthrobacter_{—}aurescens=72.3%
COD_Hybrid-Protein=68.8%

New PCT primers (Seq. 8 and 9) were constructed with reference to the known sequence for choline oxidase from Arthrobacter globiformis.

The present invention accordingly further provides an oligonucleotide, in particular a PCR primer, with one of the sequences stated in Seq. 8 or Seq. 9.

A DNA probe specific for bacterial choline oxidases was then generated by PCR.

The present invention accordingly further provides a nucleic acid, in particular DNA probe, the nucleotide sequence of which at least 85%, in particular at least 90%, particularly preferably at least 95% and very particularly preferably 100% matches the nucleotide sequence stated in Seq. 7.

The oligonucleotides or nucleic acids according to the invention are usable for identifying and/or isolating a novel choline oxidase.

Production of a hybrid choline oxidase according to the invention may proceed by using a choline oxidase according to the invention for fusion or linkage with another protein, in particular for the development of a novel enzyme, or using a nucleic acid according to the invention for fusion with another nucleic acid, in particular for the development of a novel enzyme.

The present invention further provides:

A vector which contains a nucleic acid domain according to the invention and which may be, for example, a cloning vector or an expression vector.

Moreover a host cell, which expresses or may be stimulated to express one of the proteins or derivatives according to the invention, preferably using an expression vector according to the invention. The host cell is preferably a bacterium, in particular a bacterium, which secretes the protein or derivative formed into the surrounding medium. The host cell according to the invention may belong to genus Escherichia, in particular the species Escherichia coli or the genus Bacillus, preferably the species Bacillus licheniformis, Bacillus amyloliquefaciens, Bacillus subtilis or Bacillus alcalophilus or particularly preferably the genus Arthrobacter and within this genus the species oxidans. The host cell may, however, also be a eukaryotic cell, in particular a cell, which posttranslationally modifies the protein formed.

The present invention further provides uses both of the choline oxidases according to the invention and of choline oxidases, which are already known per se but have not hitherto been taken into consideration for the claimed uses.

Such a further use provided by the present invention is accordingly the use of a choline oxidase wherein
it belongs to the family of GMC oxidoreductases,
it binds FAD as cofactor and,
at the N-terminal end of the protein, preferably at positions 20 to 25, it comprises an amino acid domain with the sequence GxGxxG, preferably with the sequence GGGSAG, wherein x denotes any desired amino acid,
as an agent, which produces hydrogen peroxide in situ.

A choline oxidase, which is preferred for the use according to the invention, is one of the choline oxidases according to the invention.

A preferred embodiment of the use according to the invention is the use for bleaching, for dye transfer inhibition and for disinfection.

The choline oxidases according to the invention and the choline oxidases usable according to the invention may advantageously be introduced into bodycare products, shampoos, hair care products, hair dyes or bleaches, oral care, tooth care or denture care products, cosmetics, detergents, cleaning agents, rinsing agents, hand washing detergents, hand dishwashing detergents, machine dishwashing detergents, disinfectants and agents for bleaching or disinfectant treatment of filter media, textiles, furs, paper, skins or leather.

A detergent or bleaching agent containing a bleaching system that is capable of producing hydrogen peroxide under the conditions of use of the agent, and optionally a synthetic surfactant, organic and/or inorganic builders and other conventional constituents of bleaching agents or detergents, wherein the bleaching system consists of a choline oxidase according to the invention and a substrate for the choline oxidase is preferred according to the invention. The substrate is preferably choline or a choline derivative, as described above.

The detergent or bleaching agent according to the invention preferably exhibits oxidase activity of 1 U/g to 20,000 U/g; it preferably presents the form of a free-flowing powder with a bulk density of 300 g/l to 1200 g/l, in particular 500 g/l to 900 g/l. Alternatively, however, it may also present the form of a pasty or liquid detergent, in particular the form of a non-aqueous liquid detergent or a non-aqueous paste, or the form of an aqueous liquid detergent or a paste which contains water.

The detergent or bleaching agent according to the invention may be packaged in an air-impermeable container, from which it is released shortly before use or during the washing operation, in particular the choline oxidase and/or the substrate for this enzyme may be enclosed in a substance which is impermeable to the enzyme and/or the substrate thereof at room temperature or in the absence of water, which substance becomes permeable to the enzyme and/or the substrate thereof under the conditions of use of the agent.

The detergent or bleaching agent according to the invention preferably contains, in addition to the bleaching system,
5 wt. % to 70 wt. %, in particular 10 wt. % to 50 wt. %, of surfactant,
10 wt. % to 65 wt. %, in particular 12 wt. % to 60 wt. %. of a water-soluble, water-dispersible inorganic builder material,
1 wt. % to 10 wt. %, in particular 2 wt. % to 8 wt. %, of water-soluble organic builder substances,
no more than 15 wt. % of solid inorganic and/or organic acids or acidic salts,
no more than 5 wt. % of heavy metal complexing agent,
no more than 5 wt. % of graying inhibitor,
no more than 5 wt. % of dye transfer inhibitor and
no more than 5 wt. % of foam inhibitor.

Due to their great industrial significance, as a complement to the particularly preferred embodiments presented above, a detailed description will now be provided of the various aspects and other constituents of detergents and cleaning agents according to the invention, i.e. characterized by the above-described choline oxidases.

A global distinction is here drawn on the basis of the material being cleaned between textiles and hard surfaces. The conditions to be selected for this purpose, in particular to be controlled by the other constituents, such as for example temperature, pH value, ionic strength, redox behavior or mechanical influences, should be optimized for the particular cleaning problem. Conventional temperatures for detergents and cleaning agents are accordingly in the range from 10° C. for manual agents through 40° C. and 60° C. up to 95° C. for machine agents or in industrial applications. Since temperature is usually continuously variable in modern washing machines and dishwashers, all intermediate temperatures are also included. The constituents of the agents in question are preferably tailored to one another. Synergistic effects with regard to cleaning performance are preferred.

A choline oxidase according to the invention may find application both in agents for major consumers or industrial users and in products for private consumers, any types of cleaning agent established in the prior art also constituting embodiments of the present invention. These include, for example, concentrates and agents to be used undiluted; for use on a commercial scale, in a washing machine or for hand washing or cleaning. These include, for example, detergents for textiles, carpets or natural fibers, these being referred to as detergents according to the present invention. These also include, for example, dishwashing detergents for dishwashers or manual dishwashing detergents or cleaning agents for hard surfaces such as metal, glass, porcelain, ceramics, glazed tiles, stone, coated surfaces, plastics, wood or leather; such products are referred to as cleaning agents according to the present invention.

Embodiments of the present invention comprise all established and/or all convenient presentations. These include, for example, solid, powdered, liquid, gel-form or pasty agents, optionally also comprising two or more phases, compressed or uncompressed; further examples include: extrudates, granules, tablets or pouches, packaged both in large containers and in portions.

The choline oxidases according to the invention are combined in agents according to the invention with, for example, one or more of the following constituents: nonionic, anionic and/or cationic surfactants, (optionally further) bleaching agents, bleach activators, bleach catalysts, builders and/or cobuilders, solvents, thickeners, sequestering agents, electrolytes, optical brighteners, graying inhibitors, corrosion inhibitors, in particular silver protection agents, soil-release active ingredients, dye transfer inhibitors, foam inhibitors, abrasives, dyes, fragrances, antimicrobial active ingredients, UV protection agents, enzymes such as for example proteases, amylases, lipases, cellulases, hemicellulases or oxidases, stabilizers, in particular enzyme stabilizers, and other components which are known from the prior art.

Preferred nonionic surfactants are alkoxylated, advantageously ethoxylated, particularly primary alcohols, preferably containing 8 to 18 carbon atoms and, on average, 1 to 12 moles of ethylene oxide (EO) per mole of alcohol, in which the alcohol radical may be linear or, preferably, methyl-branched in the 2-position or may contain linear and methyl-branched radicals in the form of the mixtures typically present in oxoalcohol radicals. Particularly preferred are, however, alcohol ethoxylates with linear radicals of alcohols of natural origin with 12 to 18 carbon atoms, e.g. from coco-, palm-, tallow- or oleyl alcohol, and an average of 2 to 8 EO per mol alcohol. Exemplary ethoxylated alcohols include C₁₂₋₁₄-alcohols with 3 EO or 4 EO, C₉₋₁₁-alcohols with 7 EO, C₃₋₁₅-alcohols with 3 EO, 5 EO, 7 EO or 8 EO, C₁₂₋₁₈-alcohols with 3 EO, 5 EO or 7 EO and mixtures thereof, such as mixtures of C₁₂₋₁₄-alcohols with 3 EO and C₁₂₋₁₈- alcohols with 5 EO. The cited carbon chain lengths and the degree of alkoxylation again constitute statistical average values that can be a whole or a fractional number for a specific product. Preferred alcohol ethoxylates have a narrowed homolog distribution (narrow range ethoxylates, NRE). In addition to these nonionic surfactants, fatty alcohols with more than 12 EO can also be used. Examples of these are tallow fatty alcohol with 14 EO, 25 EO, 30 EO or 40 EO.

A further class of preferred added nonionic surfactants, which are added either as the sole nonionic surfactant or in combination with other nonionic surfactants, is constituted by alkoxylated, preferably ethoxylated or ethoxylated and propoxylated alkyl esters of fatty acids, preferably with 1 to 4 carbon atoms in the alkyl chain, particularly fatty acid methyl esters.

A further class of nonionic surfactants, which can be advantageously used are the alkyl polyglycosides (APG). Suitable non-ionic surfactants are alkyl glycosides with the general formula RO(G)_{z} where R is a primary, linear or methyl-branched, more particularly 2-methyl-branched, aliphatic radical containing 8 to 22 and preferably 12 to 18 carbon atoms and G stands for a glycose unit containing 5 or 6 carbon atoms, preferably glucose. The degree of glycosylation z is between 1 and 4, preferably between 1.0 and 2.0 and especially between 1.1 and 1.4. Linear, alkyl polyglucosides, i.e. alkyl polyglycosides, in which the polyglycosyl radical is a glucose radical and the alkyl radical an n-alkyl radical, are preferably used.

Non-ionic surfactants of the amine oxide type, for example N-coconutalkyl-N,N-dimethylamine oxide and N-tallowalkyl-N,N-dihydroxy-ethylamine oxide, and the fatty acid alkanolamide type are also suitable. The quantity in which these non-ionic surfactants are used is preferably no more than the quantity in which the ethoxylated fatty alcohols are used and, more preferably, no more than half that quantity.

Other suitable surfactants are polyhydroxyfatty acid amides corresponding to formula (II), 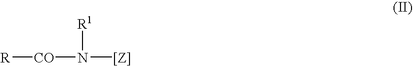
in which RCO is an aliphatic acyl group containing 6 to 22 carbon atoms, R¹ is hydrogen, an alkyl or hydroxyalkyl radical containing. 1 to 4 carbon atoms and [Z] is a linear or branched polyhydroxyalkyl group containing 3 to 10 carbon atoms and 3 to 10 hydroxyl groups. The polyhydroxy fatty acid amides are known substances, which may normally be obtained by reductive amination of a reducing sugar with ammonia, an alkylamine or an alkanolamine and subsequent acylafion with a fatty acid, a fatty acid alkyl ester or a fatty acid chloride.

The group of polyhydroxy fatty acid amides also includes compounds corresponding to formula (III), 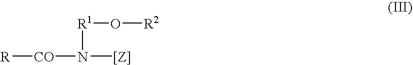
in which R is a linear or branched alkyl or alkenyl radical containing 7 to 12 carbon atoms, R¹ is a linear, branched or cyclic alkyl radical or an aryl radical containing 2 to 8 carbon atoms and R² is a linear, branched or cyclic alkyl radical or an aryl radical or an oxyalkyl radical containing 1 to 8 carbon atoms, C₁₋₄ alkyl or phenyl radicals being preferred, and [Z] is a linear polyhydroxy-alkyl radical, of which the alkyl chain is substituted by at least two hydroxyl radicals, or alkoxylated, preferably ethoxylated or propoxylated derivatives of that radical.

[Z] is preferably obtained by reductive amination of a reducing sugar, for example glucose, fructose, maltose, lactose, galactose, mannose or xylose. The N-alkoxy- or N-aryloxy substituted compounds may then be converted into the required polyhydroxy fatty acid amides by reaction with fatty acid methyl esters in the presence of an alkoxide as catalyst.

Suitable anionic surfactants are, for example, the sulfonates and sulfates. Surfactants of the sulfonate type preferably include C₉₋₁₃ alkylbenzene sulfonates, olefin sulfonates, i.e. mixtures of alkene sulfonates and hydroxyalkane sulfonates as well as disulfonates, such as those obtained from C₁₂₋₁₈ monoolefins having terminal or internal double bonds, by sulfonation with gaseous sulfur trioxide and subsequent alkaline or acidic hydrolysis of the sulfonation products. Alkane sulfonates, obtained, for example, by sulfochlorination or sulfoxidation of C₁₂₋₁₈ alkanes and subsequent hydrolysis or neutralization, are also suitable. Similarly, esters of α-sulfo fatty acids (sulfonate esters), e.g. the α-sulfonated methyl esters of hydrogenated coconut-, palm nut- or tallow fatty acids, are also suitable.

Further suitable anionic surfactants are sulfonated glycerin esters of fatty acids, including the mono-, di-, and triesters as well as their mixtures, as obtained from the esterification of a monoglycerin with 1 to 3 moles of fatty acid or from the transesterification of triglycerides with 0.3 to 2 moles of glycerin. Preferred sulfonated glycerin esters of fatty acids are the sulfonated products of saturated fatty acids with 6 to 22 carbon atoms, for example, caproic acid, caprylic acid, capric acid, myristic acid, lauric acid, palmitic acid, stearic acid or behenic acid.

Preferred alk(en)yl sulfates are the alkali and particularly the sodium salts of the sulfuric acid half esters of the C₁₂₋₁₈ fatty alcohols, for example of cocofat alcohol. Tallow fat alcohol, lauryl, myristyl, cetyl or stearyl alcohol or of the C₁₀₋₂₀ oxoalcohols and those half esters of secondary alcohols of these chain lengths. Further preferred alk(en)yl sulfates are those of the cited chain length, which comprise a synthetic straight chain alkyl radical manufactured by the petrochemical industry and which have an analogous degradation behavior to the adequate compounds from fatty chemical raw materials. The C₁₂-C₁₆ alkyl sulfates and C₁₂-C₁₅ alkyl sulfates as well as C₁₄-C₁₅ alkyl sulfates are preferred by the interests of the washing industry. The 2,3-alkyl sulfates are also suitable anionic surfactants.

Monoesters of sulfuric acid of linear or branched C₇₋₂₁ alcohols, ethoxylated with 1 to 6 mole ethylene oxide, such as 2-methyl branched C₉₋₁₁ alcohols having on average 3.5 moles ethylene oxide (EO) or C₁₂₋₁₈ fatty alcohols having 1 to 4 EO, are suitable. Because of their high foaming behavior, they are only added to detergents in relatively small amounts, for example in quantities up to 5 wt. %, normally from 1 to 5 wt. %.

Further suitable anionic surfactants are also the salts of alkyl sulfosuccinic acid—also called sulfosuccinates or esters of sulfosuccinic acid—and the monoesters and/or diesters of sulfosuccinic acid with alcohols, preferably fatty alcohols and particularly ethoxylated fatty alcohols. Preferred sulfosuccinates contain C₆₋₁₈ fatty alcohol radicals or mixtures thereof. Particularly preferred sulfosuccinates contain an alcohol radical, derived from ethoxylated fatty alcohols, considered to represent nonionic surfactants (see under). In this context, sulfosuccinates whose fatty alcohol radicals are derived from ethoxylated fatty alcohols with narrow homolog distribution, are again particularly preferred. Similarly, it is also possible to use alk(en)ylsuccinic acids having preferably 8 to 18 carbon atoms in the alk(en)yl chain or salts thereof.

Further anionic surfactants can be especially soaps. Suitable soaps are saturated fatty acid soaps, such as the salts of lauric acid, myristic acid, palmitic acid, stearic acid, hydrogenated erucic acid and behenic acid as well as soap mixtures, particularly from natural fatty acids, for example coconut, palm nut or tallow fatty acids.

The anionic surfactants including the soaps can be present in the form of their sodium, potassium or ammonium salts as well as soluble salts of organic bases, such as mono-, di- and triethanolamine. Preferably, the anionic surfactants are present in the form of their sodium or potassium salts, particularly in the form of their sodium salts.

The cleaning or washing agents according to the invention can comprise the surfactants in amounts from preferably 5 wt. % to 50 wt. %, particularly from 8 wt. % to 30 wt. %, based on the finished agent.

In addition, the agents according to the invention can comprise bleaches. Among the compounds, which serve as bleaches and liberate H₂O₂ in water, sodium percarbonate, sodium perborate tetrahydrate and sodium perborate monohydrate are of particular importance. Examples of further bleaches, which may be used, are peroxypyrophosphates, citrate perhydrates and H₂O₂-liberating peracidic salts or peracids, such as persulfates or persulfuric acid. The urea peroxyhydrate, percarbamide, H₂N—CO—NH₂.H₂O₂ is also suitable. Particularly when agents are used to clean hard surfaces, for example for automatic dishwashers, they can, if desired, also comprise bleaches from the group of organic bleaches, although in principal they can also be used for washing textiles. Typical organic bleaches are the diacyl peroxides, such as, for example, dibenzoyl peroxide. Further typical organic bleaches that can be used are the peroxy acids, particular examples being the peroxybenzoic acids and their ring-substituted derivatives, such as alkylperoxybenzoic acids, but also peroxy-α-naphthoic acid and magnesium monoperphthalate, the aliphatic or substituted aliphatic peroxy acids, such as peroxylauric acid, peroxystearic acid, ε-phthalimidoperoxycaproic acid [phthaloiminoperoxyhexanoic acid (PAP)], o-carboxybenzamido peroxycaproic acid, N-nonenylamidoperadipic acid and N-nonenylamido persuccinates, and aliphatic and araliphatic peroxydicarboxylic acids, such as 1,12-diperoxycarboxylic acid, 1,9-diperoxyazelaic acid, diperoxysebacic acid, diperoxybrassylic acid, the diperoxyphthalic acids, 2-decyldiperoxybutane-1,4-dioic acid, N,N-terephthaloyl-di(6-aminopercaproic acid).

The content of bleach in the agent can range from 1 to 40 wt. % and particularly 10 to 20 wt. %, wherein perborate monohydrate or percarbonate is advantageously added. A synergistic utilization of amylase with percarbonate or of amylase with percarboxylic acids has been disclosed in the applications WO 99/63036 and WO 99/63037.

When the agents are used at temperatures of 60° C. and below, and particularly during the prewash, they can comprise bleach activators in order to achieve an improved bleaching action. Bleach activators, which can be used are compounds which, under perhydrolysis conditions, produce aliphatic peroxycarboxylic acids having preferably 1 to 10 carbon atoms, in particular 2 to 4 carbon atoms, and/or optionally substituted perbenzoic acid. Substances, which carry O-acyl and/or N-acyl groups of said number of carbon atoms and/or optionally substituted benzoyl groups, are suitable. Preference is given to polyacylated alkylenediamines, in particular tetraacetyl ethylenediamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenolsulfonates, in particular n-nonanoyl- or isononanoyloxybenzenesulfonate (n- or iso-NOBS), acylated hydroxycarboxylic acids, such as triethyl-O-acetyl citrate (TEOC), carboxylic acid anhydrides, in particular phthalic anhydride, isatoic anhydride and/or succinic anhydride, amides of carboxylic acids, such as N-methyldiacetamide, glycolide, acylated polyhydric alcohols, in particular triacetin, ethylene glycol diacetate, isopropenyl acetate, 2,5-diacetoxy-2,5-dihydrofuran and the enol esters disclosed in DE 196 16 693 and DE 196 16 767 as well as acylated sorbitol and mannitol or their mixtures disclosed in European Patent application EP 0 525 239 (SORMAN), acylated sugar derivatives, particularly pentaacetyl glucose (PAG), pentaacetyl fructose, tetraacetyl xylose and octaacetyl lactose, and acetylated, optionally N-alkylated, glucamine and gluconolactone, triazole or triazole derivatives and/or particulate caprolactams and/or caprolactam derivatives, preferred N-acylated lactams, for example N-benzoyl caprolactam and N-acetyl caprolactam, which are known from the Patent applications WO 94/27970, WO 94/28102, WO 95/00626, WO 95/14759 and WO 95/17498. Similarly, the hydrophilic substituted acyl acetals disclosed in the German Patent application DE 196 16 769 and acyl lactams described in the German Patent application DE 196 16 770 and the international Patent application WO 95/14075 are also preferably used. Similarly, the nitrile derivatives, such as cyano pyridines, nitrile quats, for example N-alkylammonium acetonitrile, and/or cyanamide derivatives can also be used. Preferred bleach activators are sodium 4-(octanoyl)-benzene sulfonate, n-nonanoyl- or isononanoyloxybenzene sulfonate (n- or iso-NOBS), undecenoyloxybenzene sulfonate (DOBS), decanoyloxybenzoic acid (DOBA, OBC 10) and/or dodecanoyloxybenzene sulfonate (OBS 12), as well as N-methylmorpholinium acetonitrile (MMA). These types of bleach activators can be comprised in the typical quantity range of 0.01 to 20 wt. %, preferably in quantities of 0.1 to 15 wt. %, particularly 1 wt. % to 10 wt. %.

In addition to the conventional bleach activators, or instead of them, so-called bleach catalysts may also be incorporated into the agents according to the invention. These substances are bleach-boosting transition metal salts or transition metal complexes, such as, for example, Mn-, Fe-, Co-, Ru- or Mo-salen complexes or -carbonyl complexes. Mn-, Fe-, Co-, Ru-, Mo-, Ti-, V- and Cu-complexes with N-containing tripod ligands, and Co-, Fe-, Cu- and Ru-ammine complexes can also be used as bleach catalysts, whereby it is preferred to add such compounds, which are described in DE 197 09 284 A1.

Acetonitrile derivatives according to WO 99/63038 and compounds of bleach activating transition metal complexes according to WO 99/63041 in combination with amylases are also able to develop a bleach activating action.

Agents according to the invention generally comprise one or more builders, particularly zeolites, silicates, carbonates, organic cobuilders and also—where there are no ecological reasons preventing their use—phosphates. The last are preferred builders, particularly when added to automatic dishwasher detergents.

Here may be cited crystalline, layered sodium silicates corresponding to the general formula NaMSiₓO₂ₓ₊₁.yH₂O, wherein M is sodium or hydrogen, x is a number from 1.9 to 4 and y is a number from 0 to 20, preferred values for x being 2, 3 or 4. Such crystalline layered silicates are described, for example in the European Patent application EP 0 164 514. Preferred crystalline, layered silicates of the given formula are those in which M stands for sodium and x assumes the values 2 or 3. Both β- and δ-sodium disilicates Na₂Si₂O₅.yH₂O are particularly preferred. These compounds are commercially available, for example under the denomination SKS® (Clariant). SKS-6® is predominantly a 6-sodium disilicate with the formula Na₂Si₂O₅-yH₂O, SKS-7® is predominantly a β-sodium silicate. On reaction with acids (e.g. citric acid or carbonic acid), 6-sodium disilicate yields Kanemite NaHSi₂O₅.yH₂O, which is commercially available under the designations SKS-9® and SKS-10® from Clariant. It can also be advantageous to initiate chemical modifications of these layered silicates. Thus, the alkalinity, for example, of the layered silicates can be suitably influenced. Layered silicates modified with phosphate or with carbonate have a modified crystal morphology in comparison to δ-sodium disilicate, dissolve faster and in comparison to δ-sodium disilicate show a higher calcium binding potential. Thus, layered silicates of the general formula Na₂O ●y SiO₂●z P₂O₅′, in which the ratio x to y corresponds to a number 0.35 to 0.6, the ratio x to z corresponds to a number from 1.75 to 1200 and the ratio y to z a number from 4 to 2800, are described in the Patent application DE 196 01 063. The solubility of the layered silicates can also be increased by the use of particularly finely dispersed layered silicates. Compounds of crystalline, layered silicates with other ingredients can also be used. Compounds with cellulose derivatives, which possess advantages in the disintegration action, and which are particularly used in washing agent tablets, as well as compounds with polycarboxylates, for example citric acid or polymeric polycarboxylates, for example copolymers of acrylic acid can be particularly cited in this context.

Other useful builders are amorphous sodium silicates with a modulus (Na₂O: SiO₂ ratio) of 1:2 to 1:3.3, preferably 1:2 to 1:2.8 and more preferably 1:2 to 1:2,6, which dissolve with a delay and exhibit multiple wash cycle properties. The delay in dissolution compared with conventional amorphous sodium silicates can have been obtained in various ways, for example by surface treatment, compounding, compressing/compacting or by over-drying. In the context of the invention, the term “amorphous” is also understood to encompass “X-ray amorphous”. In other words, the silicates do not produce any of the sharp X-ray reflexes typical of crystalline substances in X-ray diffraction experiments, but at best one or more maxima of the scattered X-radiation, which have a width of several degrees of the diffraction angle. However, particularly good builder properties may even be achieved where the silicate particles produce indistinct or even sharp diffraction maxima in electron diffraction experiments. This can be interpreted to mean that the products have microcrystalline regions between 10 and a few hundred nm in size, values of up to at most 50 nm and especially up to at most 20 nm being preferred. This type of X-ray amorphous silicates similarly possesses a delayed dissolution in comparison with the customary water glasses. Compacted/densified amorphous silicates, compounded amorphous silicates and over dried X-ray-amorphous silicates are particularly preferred.

Of the suitable fine crystalline, synthetic zeolites containing bound water, zeolite A and/or P are preferred. A particularly preferred zeolite P is zeolite MAP® (a commercial product of Crosfield). However, the zeolites X as well as mixtures of A, X and/or P are also suitable. Commercially available and preferred in the context of the present invention is, for example, also a co-crystallizate of zeolite X and zeolite A (ca. 80 wt. % zeolite X), which is marketed under the name of VEGOBOND AX® by Condea Augusta S.p.A. and which can be described by the Formula
nNa₂O.(1−n)K₂O.Al₂O₃.(2−2,5)SiO₂.(3,5−5,5)H₂O

Suitable zeolites have a mean particle size of less than 10 μm (volume distribution, as measured by the Coulter Counter Method) and contain preferably 18 to 22% by weight and more preferably 20 to 22% by weight of bound water.

Naturally, the generally known phosphates can also be added as builders, in so far that their use should not be avoided on ecological grounds. In the washing and cleaning agent industry, among the many commercially available phosphates, the alkali metal phosphates, particularly pentasodiumu or pentaalkalium triphosphates (sodium or potassium tripolyphosphate) are particularly preferred.

“Alkali metal phosphates” is the collective term for the alkali metal (more particularly sodium and potassium) salts of the various phosphoric acids, including metaphosphoric acids (HPO₃)ₙ and orthophosphoric acid (H₃PO₄) and representatives of higher molecular weight. The phosphates combine several advantages: they act as alkalinity sources, prevent lime deposits on machine parts and lime incrustations in fabrics and, in addition, contribute towards the cleaning effect.

“Alkali metal phosphates” is the collective term for the alkali metal (more particularly sodium and potassium) salts of the various phosphoric acids, including metaphosphoric acids (HPO₃)ₙ and orthophosphoric acid (H₃PO₄) and representatives of higher molecular weight. The phosphates combine several advantages: they act as alkalinity sources, prevent lime deposits on machine parts and lime incrustations in fabrics and, in addition, contribute towards the cleaning effect.

Sodium dihydrogen phosphate NaH₂PO₄ exists as the dihydrate (density 1.91 gcm⁻³, melting point 60° C.) and as the monohydrate (density 2.04 gcm⁻³). Both salts are white, readily water-soluble powders that on heating, lose the water of crystallization and at 200° C. are converted into the weakly acidic diphosphate (disodium hydrogen diphosphate, Na₂H₂P₂O₇) and, at higher temperatures into sodium trimetaphosphate (Na₃P₃O₉) and Maddrell's salt (see below). NaH₂PO₄ shows an acidic reaction. It is formed by adjusting phosphoric acid with sodium hydroxide to a pH value of 4.5 and spraying the resulting “mash”. Potassium dihydrogen phosphate (primary or monobasic potassium phosphate, potassium biphosphate, KDP), KH₂PO₄, is a white salt with a density of 2.33 gcm⁻³, has a melting point of 253° C. [decomposition with formation of potassium polyphosphate (KPO₃)ₓ] and is readily soluble in water.

Disodium hydrogen phosphate (secondary sodium phosphate), Na₂HPO₄, is a colorless, readily water-soluble crystalline salt. It exists in anhydrous form and with 2 mol (density 2.066 gcm⁻³, water loss at 95° C.), 7 mol (density 1.68 gcm⁻³, melting point 48° C. with loss of 5H₂O) and 12 mol of water (density 1.52 gcm⁻³, melting point 35° C. with loss of 5H₂O), becomes anhydrous at 100° C. and, on fairly intensive heating, is converted into the diphosphate Na₄P₂O₇. Disodium hydrogen phosphate is prepared by neutralization of phosphoric acid with soda solution using phenolphthalein as indicator. Dipotassium hydrogen phosphate (secondary or dibasic potassium phosphate), K₂HPO₄, is an amorphous white salt, which is readily soluble in water.

Trisodium phosphate, tertiary sodium phosphate, Na₃PO₄, consists of colorless crystals with a density of 1.62 gcm⁻³ and a melting point of 73-76° C. (decomposition) as the dodecahydrate, a melting point of 100° C. as the decahydrate (corresponding to 19-20% P₂O₅) and a density of 2.536 gcm⁻³ in anhydrous form (corresponding to 39-40% P₂O₅). Trisodium phosphate is readily soluble in water through an alkaline reaction and is prepared by concentrating a solution of exactly 1 mole of disodium phosphate and 1 mole of NaOH by evaporation. Tripotassium phosphate (tertiary or tribasic potassium phosphate), K₃PO₄, is a white deliquescent granular powder with a density of 2.56 gcm⁻³, has a melting point of 1340° C. and is readily soluble in water through an alkaline reaction. It is formed, for example, when Thomas slag is heated with coal and potassium sulfate. Despite their higher price, the more readily soluble and therefore highly effective potassium phosphates are often preferred to corresponding sodium compounds in the detergent industry.

Tetrasodium diphosphate (sodium pyrophosphate), Na₄P₂O₇, exists in anhydrous form (density 2.534 gcm⁻³, melting point 988° C., a figure of 880° C. has also been mentioned) and as the decahydrate (density 1.815-1.836 gcm⁻³, melting point 94° C. with loss of water). Both substances are colorless crystals, which dissolve in water through an alkaline reaction. Na₄P₂O₇ is formed when disodium phosphate is heated to more than 200° C. or by reacting phosphoric acid with soda in a stoichiometric ratio and spray drying the solution. The decahydrate complexes heavy metal salts and hardness salts and, hence, reduces the hardness of water. Potassium diphosphate (potassium pyrophosphate), K₄P₂O₇, exists in the form of the trihydrate and is a colorless hygroscopic powder with a density of 2.33 gcm^{-3, which} is soluble in water, the pH of a 1% solution at 25° C. being 10.4.

Relatively high molecular weight sodium and potassium phosphates are formed by condensation of NaH₂PO₄ or KH₂PO₄. They may be divided into cyclic types, namely the sodium and potassium metaphosphates, and chain types, the sodium and potassium polyphosphates. The chain types in particular are known by various different names: fused or calcined phosphates, Graham's salt, Kurrol's salt and Maddrell's salt. All higher sodium and potassium phosphates are known collectively as condensed phosphates.

The industrially important pentasodium triphosphate, Na₅P₃O₁₀ (sodium tripolyphosphate), is anhydrous or crystallizes with 6H₂O to a non-hygroscopic white water-soluble salt which and which has the general formula NaO—[P(O)(ONa)—O]ₙ—Na where n=3. Around 17 g of the salt free from water of crystallization dissolve in 100 g of water at room temperature, around 20 g at 60° C. and around 32 g at 100° C. After heating the solution for 2 hours to 100° C., around 8% orthophosphate and 15% diphosphate are formed by hydrolysis. In the preparation of pentasodium triphosphate, phosphoric acid is reacted with soda solution or sodium hydroxide in a stoichiometric ratio and the solution is spray-dried. Similarly to Graham's salt and sodium diphosphate, pentasodium triphosphate dissolves many insoluble metal compounds (including lime soaps, etc.). Pentapotassium triphosphate, K₅P₃O₁₀ (potassium tripolyphosphate), is marketed for example in the form of a 50% by weight solution (>23% P₂O₅, 25% K₂O). The potassium polyphosphates are widely used in the detergent industry. Sodium potassium tripolyphosphates, which may also be used in accordance with the present invention, also exist. They are formed for example when sodium trimetaphosphate is hydrolyzed with KOH:
(NaPO₃)₃+2 KOH→Na₃K₂P₃O₁₀+H₂O

According to the invention, they may be used in exactly the same way as sodium tripolyphosphate, potassium tripolyphosphate or mixtures thereof. Mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate may also be used in accordance with the invention.

Organic cobuilders, which may be used in the washing and cleaning agents according to the invention, include, in particular, polycarboxylates/polycarboxylic acids, polymeric polycarboxylates, aspartic acid, polyacetals, dextrins, other organic cobuilders (see below) and phosphonates. These classes of substances are described below.

Useful organic builders are, for example, the polycarboxylic acids usable in the form of their sodium salts, polycarboxylic acids, which in this context being understood to be carboxylic acids that carry more than one acid function. These include, for example, citric acid, adipic acid, succinic acid, glutaric acid, malic acid, tartaric acid, maleic acid, fumaric acid, sugar acids, aminocarboxylic acids, nitrilotriacetic acid (NTA), providing its use is not ecologically unsafe, and mixtures thereof. Preferred salts are the salts of the polycarboxylic acids, such as citric acid, adipic acid, succinic acid, glutaric acid, tartaric acid, sugar acids and mixtures thereof.

The acidsper se may also be used. Besides their building effect, the acids also typically have the property of an acidifying component and, hence also serve to establish a relatively low and mild pH in washing or cleaning agents, in so far that the pH, which results from the mixture of other components is not wanted. Acids that are system-compatible and environmentally compatible such as citric acid, acetic acid, tartaric acid, malic acid, glycolic acid, succinic acid, glutaric acid, adipic acid, gluconic acid and mixtures thereof are particularly mentioned in this regard. However, mineral acids, particularly sulfuric acid or bases, particularly ammonium or alkali hydroxides can also serve as pH regulators. Such regulators are comprised in the agents according to the invention in amounts preferably not above 20 wt. %, particularly from 1.2 wt. % to 17 wt. %.

Other suitable builders are polymeric polycarboxylates, i.e. for example, the alkali metal salts of polyacrylic or polymethacrylic acid, for example those with a relative molecular weight of 500 to 70 000 g/mol.

The molecular weights mentioned in this specification for polymeric polycarboxylates are weight-average molecular weights Mw of the particular acid form which, fundamentally, were determined by gel permeation chromatography (GPC), equipped with a UV detector. The measurement was carried out against an external polyacrylic acid standard, which provides realistic molecular weight values by virtue of its structural similarity to the polymers investigated. These values differ distinctly from the molecular weights measured against polystyrene sulfonic acids as standard. The molecular weights measured against polystyrene sulfonic acids are generally higher than the molecular weights mentioned in this specification.

Particularly suitable polymers are polyacrylates, which preferably have a molecular weight of 2000 to 20 000 g/mol. By virtue of their superior solubility, preferred representatives of this group are the short-chain polyacrylates, which have molecular weights of 2000 to 10 000 g/mol and, more particularly, 3000 to 5000 g/mol.

Also suitable are copolymeric polycarboxylates, particularly those of acrylic acid with methacrylic acid and those of acrylic acid or methacrylic acid with maleic acid. Acrylic acid/maleic acid copolymers containing 50 to 90% by weight of acrylic acid and 50 to 10% by weight of maleic acid have proved to be particularly suitable. Their relative molecular weights, based on the free acids, are generally in the range from 2000 to 70 000 g/mol, preferably in the range from 20 000 to 50 000 g/mol and more preferably in the range from 30 000 to 40 000 g/mol. The (co)polymeric polycarboxylates may be used either in powder form or in the form of an aqueous solution. The content of (co)polymeric polycarboxylates in the detergents is preferably 0.5 to 20% by weight and more particularly 1 to 10% by weight.

To increase the water-solubility, the polymers can also comprise allylsulfonic acids as monomers, for example allyloxybenzene sulfonic acid and methallyl sulfonic acid.

Other particularly preferred polymers are biodegradable polymers of more than two different monomer units, for example those which contain salts of acrylic acid and maleic acid and vinyl alcohol or vinyl alcohol derivatives as monomers or those which contain salts of acrylic acid and 2-alkylallyl sulfonic acid and sugar derivatives as monomers.

Other preferred copolymers are those, which preferably contain acrolein and acrylic acid/acrylic acid salts or acrolein and vinyl acetate as monomers.

Other preferred builders are polymeric aminodicarboxylic acids, salts or precursors thereof. Polyaspartic acids or salts and derivatives thereof are particularly preferred.

Other suitable builders are polyacetals, which may be obtained by reaction of dialdehydes with polyol carboxylic acids containing 5 to 7 carbon atoms and at least three hydroxyl groups. Preferred polyacetals are obtained from dialdehydes, such as glyoxal, glutaraldehyde, terephthalaldehyde and mixtures thereof and from polyol carboxylic acids, such as gluconic acid and/or glucoheptonic acid.

Other suitable organic builders are dextrins, for example oligomers or polymers of carbohydrates, which may be obtained by partial hydrolysis of starches. The hydrolysis may be carried out by standard methods, for example acid- or enzyme-catalyzed methods. The end products are preferably hydrolysis products with average molecular weights of 400 to 500 000 g/mol. A polysaccharide with a dextrose equivalent (DE) of 0.5 to 40 and, more particularly, 2 to 30 is preferred, the DE being an accepted measure of the reducing effect of a polysaccharide by comparison with dextrose which has a DE of 100. Both maltodextrins with a DE of 3 to 20 and dry glucose syrups with a DE of 20 to 37 and also so-called yellow dextrins and white dextrins with relatively high molecular weights of 2000 to 30 000 g/mol may be used.

The oxidized derivatives of such dextrins are their reaction products with oxidizing agents that are capable of oxidizing at least one alcohol function of the saccharide ring to the carboxylic acid function. Particularly preferred organic builders for the agents according to the invention are oxidized starches or their derivatives from the applications EP 472 042, WO 97/25399 and EP 755 944.

Other suitable cobuilders are oxydisuccinates and other derivatives of disuccinates, preferably ethylenediamine disuccinate. Ethylenediamine-N,N′-disuccinate (EDDS) is preferably used in the form of its sodium or magnesium salts. Glycerol disuccinates and glycerol trisuccinates are also preferred in this connection. The quantities used in zeolite-containing and/or silicate-containing formulations are from 3 to 15% by weight. −%.

Other useful organic cobuilders are, for example, acetylated hydroxycarboxylic acids and salts thereof which may optionally be present in lactone form and which contain at least 4 carbon atoms, at least one hydroxy group and at most two acid groups.

Another class of substances with cobuilder properties are the phosphonates, more particularly hydroxyalkane and aminoalkane phosphonates. Among the hydroxyalkane phosphonates, 1-hydroxyethane-1,1-diphosphonate (HEDP) is particularly important as a cobuilder. It is preferably used in the form of the sodium salt, the disodium salt showing a neutral reaction and the tetrasodium salt an alkaline reaction (pH 9). Preferred aminoalkane phosphonates are ethylenediamine tetramethylene phosphonate (EDTMP), diethylenetriamine pentamethylenephosphonate (DTPMP) and higher homologs thereof. They are preferably used in the form of the neutrally reacting sodium salts, for example as the hexasodium salt of EDTMP or as the hepta- and octasodium salts of DTPMP. Of the phosphonates, HEDP is preferably used as a builder. In addition, the aminoalkane phosphonates have a pronounced heavy metal binding capacity. Accordingly, it can be of advantage, particularly where the agents also contain bleach, to use aminoalkane phosphonates, more particularly DTPMP, or mixtures of the phosphonates mentioned.

In addition, any compounds capable of forming complexes with alkaline earth metal ions may be used as cobuilders.

The agents according to the invention can optionally comprise builders in quantities of up to 90 wt. % and preferably in amounts up to 75 wt. %. Washing agents according to the invention have builder contents particularly from 5 wt. % to 50 wt. %. In agents according to the invention for the cleaning of hard surfaces, particularly for automatic cleaning of tableware, the content of builder substances ranges from 5 wt. % to 88 wt. %, wherein advantageously, no water-insoluble builder materials are added to such agents. In a preferred embodiment, the agent according to the invention, particularly for automatic dishwashers, comprises 20 wt. % to 40 wt. % of water-soluble organic builders, particularly alkali citrate, 5 wt. % to 15 wt. % alkali carbonate and 20 wt. % to 40 wt. % alkali disilicate.

Solvents that can be added to the liquid to gel-like compositions of washing and cleaning agents originate from the group of mono- or polyhydric alcohols, alkanolamines or glycol ethers, in so far that they are miscible with water in the defined concentrations. Preferably, the solvents are selected from ethanol, n- or i-propanol, butanols, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl-, -ethyl- or -propyl ether, dipropylene glycol methyl-, or -ethyl ether, methoxy-, ethoxy- or butoxy triglycol, 1-butoxyethoxy-2-propanol, 3-methyl-3-methoxybutanol, propylene glycol t-butyl ether as well as mixtures of these solvents.

Solvents can be added to the liquid to gel-like washing and cleaning agents in amounts between 0.1 and 20 wt. %, preferably, however below 15 wt. % and particularly below 10 wt. %.

One or more thickeners or thickener systems can be added to the compositions according to the invention to adjust the viscosity. These high molecular weight substances, which are also called swelling agents, soak up mostly liquids, thereby swelling up and subsequently transform into viscous, real or colloidal solutions.

Suitable thickeners are inorganic or polymeric organic compounds. The inorganic thickeners include, for example, polysilicic acids, clays such as, montmorillonite, zeolites, silicas and bentonite. The organic thickeners are derived from the group of natural polymers, modified natural polymers and synthetic polymers. Those natural polymers are, for example, agar agar, guar gum, gum arabic, alginates, pectins, polyoses, xanthane gum, karaya gum, locust bean flour, starches and celluloses. Examples can be cited as carboxymethyl cellulose and other cellulose ethers, hydroxyethyl- and hydroxypropyl cellulose as well as flour ether. Fully synthetic thickeners are polymers such as polyacrylics and polymethacrylics, vinyl polymers, polycarboxylic acids, polyethers, polyimines, polyamides and polyurethanes.

The thickeners can be comprised in amounts up to 5 wt. %, preferably from 0.05 to 2 wt. %, and particularly preferably from 0.1 to 1.5 wt. %, based on the finished preparation.

The washing or cleaning agents according to the invention can optionally comprise other ingredients, such as sequestering agents, electrolytes and further auxiliaries.

The washing agents for textiles may contain derivatives of diaminostilbene disulfonic acid or alkali metal salts thereof as optical brighteners. Suitable optical brighteners are, for example, salts of 4,4′-bis-(2-anilino-4-morpholino-1,3,5-triazinyl-6-amino)stilbene-2,2′-disulfonic acid or compounds of similar structure which contain a diethanolamino group, a methylamino group and anilino group or a 2-methoxyethylamino group instead of the morpholino group. Brighteners of the substituted diphenylstyryl type, for example alkali metal salts of 4,4′-bis-(2-sulfostyryl)diphenyl, 4,4′-bis(4-chloro-3-sulfostyryl)diphenyl or 4-(4-chlorostyryl)-4′-(2-sulfostyryl)diphenyl, may also be present. Mixtures of the brighteners mentioned may also be used.

Graying inhibitors have the function of keeping the dirt detached from the fiber in suspension in the liquor. Suitable for this purpose are water-soluble colloids, usually organic in nature, examples being starch, glue, gelatins, salts of ether carboxylic acids or ether sulfuric acids of the starch or the celluloses, or salts of acidic sulfuric acid esters of cellulose or of starch. Water-soluble polyamides containing acidic groups are also suitable for this purpose. In addition, soluble starch preparations and starch products other than those mentioned above may be used, examples being aldehyde starches. Preference, however, is given to the use of cellulose ethers such as carboxymethyl cellulose (Na salt), methyl cellulose, hydroxyalkyl cellulose, and mixed ethers such as methyl hydroxyethyl cellulose, methyl hydroxypropyl cellulose, methyl carboxymethyl cellulose and mixtures thereof, which can be added, for example in amounts of 0.1 to 5 wt. %, based on the agent.

In order to realize a silver corrosion protection, silver protectors can be added to the cleaning agents for tableware according to the invention. Benzotriazoles, ferric chloride or CoSO₄, for example are known from the prior art. As is known from the European Patent EP 0 736 084 B1, for example, particularly suitable silver protectors for general use with enzymes are salts and/or complexes of manganese, titanium, zirconium, hafnium, vanadium, cobalt or cerium, in which the cited metals exist in the valence states II, III, IV, V or VI. Examples of these types of compounds are MnSO₄, V₂O₅, V₂O₄, VO₂, TiOSO₄, K₂TiF₆, K₂ZrF₆, Co(NO₃)₂, Co(NO₃)₃ and mixtures thereof.

“Soil-release” active substances or “soil repellants” are mainly polymers, which on being used in a washing agent furnish dirt-repellant properties to the wash fibers and/or reinforce the dirt release power of the normal washing agent ingredients. A comparable effect can also be observed by their use in cleaning agents for hard surfaces.

Particularly efficient and long-known soil-release agents are copolyesters with dicarboxylic acid, alkylene glycol and polyalkylene glycol units. Examples of these are copolymers or mixed polymers of polyethylene terephthalate and polyoxyethylene glycol (DT 16 17 141 or DT 22 00 911). German Offenlegungsschrift DT 22 53 063 cites acid agents, which inter alia comprise a copolymer of a dibasic acid and an alkylene or cycloalkylene polyglycol. Polymers of ethylene terephthalate and polyethylene oxide terephthalate and their use in washing agents are described in the German texts DE 28 57 292 and DE 33 24 258 and the European Patent EP 0 253 567. The European Patent EP 066 944 relates to agents, which contain a copolyester of ethylene glycol, polyethylene glycol, aromatic dicarboxylic acids and sulfonated aromatic dicarboxylic acids in defined molar ratios. Polyesters, end-capped with methyl or ethyl groups, with ethylene and/or propylene terephthalate units and polyethylene oxide terephthalate units and washing agents that comprise such a soil-release polymer are known from EP 0 185 427. The European Patent EP 0 241 984 relates to a polyester, which beside oxyethylene groups and terephthalic acid units also comprises substituted ethylene units as well as glycerin units. Polyesters are known from EP 0 241 985 which contain, beside oxyethylene groups and terephthalic acid units, 1,2-propylene, 1,2-butylene and or 3-methoxy-1,2-propylene groups as well as glycerin, and are end-capped with C₁ to C₄ alkyl groups. Polyesters with polypropylene terephthalate units and polyoxyethylene terephthalate units, at least partially end-capped with C₁₋₄ alkyl or acyl radicals are known from the European Patent application EP 0 272 033. The European Patent EP 0 274 907 describes soil-release polyesters containing terephthalate end-capped with sulfoethyl groups. According to the European Patent application EP 0 357 280, soil-release polyesters with terephthalate units, alkylene glycol units and poly-C₂₋₄ glycol units are manufactured by sulfonation of the unsaturated end groups. The international Patent application WO 95/32232 relates to acid, aromatic dirt-repellant polyesters. Non-polymeric soil-repellants with a plurality of functional units are known from the international Patent application WO 97/31085 for cotton materials: a first unit, which can be cationic, for example, is able to be adsorbed onto the cotton surface by electrostatic attraction, and a second unit, which is designed to be hydrophobic, is responsible for the retention of the active agent at the water/cotton interface.

Color transfer inhibitors that can be used in washing agents for textiles according to the invention particularly include polyvinyl pyrrolidones, polyvinyl imidazoles, polymeric N-oxides such as polyvinyl pyridine-N-oxide and copolymers of vinyl pyrrolidone with vinyl imidazole.

On using the agents in automatic cleaning processes, it can be advantageous to add foam inhibitors. Suitable foam inhibitors include for example, soaps of natural or synthetic origin, which have a high content of C₁₆-C₂₄ fatty acids. Suitable non-surface-active types of foam inhibitors are, for example, organopolysiloxanes and mixtures thereof with microfine, optionally silanised silica and also paraffins, waxes, microcrystalline waxes and mixtures thereof with silanised silica or bis-fatty acid alkylenediamides such as bis-stearyl ethylenediamide. Mixtures of various foam inhibitors, for example mixtures of silicones, paraffins or waxes, are also used with advantage. Preferably, the foam inhibitors, particularly silicone and/or paraffin-containing foam inhibitors are loaded onto a granular, water-soluble or dispersible carrier material. In this case, mixtures of paraffins and bis stearylethylene diamides are preferred.

Furthermore, a cleaning agent for hard surfaces according to the invention can comprise ingredients having an abrasive action, particularly from the group including quartz powder, wood flour, plastic powder, chalk and glass microspheres as well as mixtures thereof. The cleaning agents according to the invention comprise preferably not more than 20 wt. %, particularly from 5 wt. % to 15 wt. % of abrasive materials.

Colorants and fragrances may be added to the washing or cleaning agents in order to improve the aesthetic impression created by the products and to provide the consumer not only with the required performance but also with a visually and sensorially “typical and unmistakable” product. Suitable perfume oils or fragrances include individual perfume compounds, for example synthetic products of the ester, ether, aldehyde, ketone, alcohol and hydrocarbon type. Perfume compounds of the ester type are, for example, benzyl acetate, phenoxyethyl isobutyrate, p-tert.-butylcyclohexyl acetate, linalyl acetate, dimethylbenzyl carbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether; the aldehydes include, for example, the linear alkanals containing 8 to 18 carbon atoms, citral, citronellal, citronellyloxyacetaldehyde, cyclamen aldehyde, hydroxycitronellal, lilial and bourgeonal; the ketones include, for example, the ionones, α-isomethyl ionone and methyl cedryl ketone; the alcohols include anethol, citronellol, eugenol, geraniol, linalool, phenylethyl alcohol and terpineol and the hydrocarbons include, above all, the terpenes, such as limonene and pinene. However, mixtures of various perfumes, which together produce an attractive perfume note, are preferably used. Perfume oils such as these may also contain natural perfume mixtures obtainable from vegetal sources, for example pine, citrus, jasmine, patchouli, rose or ylang-ylang oil. Also suitable are muscatel oil, oil of sage, chamomile oil, clove oil, melissa oil, mint oil, cinnamon leaf oil, lime blossom oil, juniper berry oil, vetivert oil, olibanum oil, galbanum oil and ladanum oil and orange blossom oil, neroli oil, orange peel oil and sandalwood oil. Normally the content of dyes lies below 0.01 wt. %, while fragrances can make up to 2 wt. % of the total formulation of the washing and cleaning agents.

The fragrances may be directly incorporated in the agents, although it can also be of advantage to apply the fragrances on carriers, which reinforce the adsorption of the perfume on the washing and thereby ensuring a long-lasting fragrance on the textiles by decreasing the release of the fragrance. Suitable carrier materials have proved to be for example, cyclodextrins, the cyclodextrin/perfume complexes optionally being coated with other auxiliaries. A further preferred carrier for fragrances is the already described zeolite X, which can also take up fragrances instead of, or in mixtures with surfactants. Accordingly, preferred washing and cleaning agents comprise the described zeolite X and fragrances, which are preferably at least partially absorbed on the zeolite.

Preferred colorants, which are not difficult for the expert to choose, have high storage stability, are not affected by the other ingredients of the detergents or by light and do not have any pronounced substantivity for the textile fibers being treated, so as not to color them.

To control microorganisms, the cleaning agents may contain antimicrobial agents. Depending on the antimicrobial spectrum and the action mechanism, antimicrobial agents are classified as bacteriostatic agents and bactericides, fungistatic agents and fungicides, etc. Important representatives of these groups are, for example, benzalkonium chlorides, alkylaryl sulfonates, halophenols and phenol mercuric acetate. In the present context of the invntive teaching according to the invention, the expressions “antimicrobial activity” and “antimicrobial agent” have the usual technical meanings as defined, for example, by K. H. Wallhäusser in “Praxis der Sterilisation, Desinfektion—Konservierung Keimidentifizierung—Betriebshygiene” (5th Edition, Stuttgart/New York: Thieme, 1995), any of the substances with antimicrobial activity described therein being usable. Suitable antimicrobial agents are preferably selected from the groups of alcohols, amines, aldehydes, antimicrobial acids and salts thereof, carboxylic acid esters, acid amides, phenols, phenol derivatives, diphenyls, diphenylalkanes, urea derivatives, oxygen and nitrogen acetals and formals, benzamidines, isothiazolines, phthalimide derivatives, pyridine derivatives, antimicrobial surface-active compounds, guanidines, antimicrobial amphoteric compounds, quinolines, 1,2-dibromo-2,4-dicyanobutane, iodo-2-propyl butyl carbamate, iodine, iodophores, peroxy compounds, halogen compounds and mixtures of the above.

Consequently, the antimicrobial active substances can be chosen among ethanol, n-propanol, i-propanol, 1,3-butanediol, phenoxyethanol, 1,2-propylenelycol, glycerin, undecylenic acid, benzoic acid, salicylic acid, dihydracetic acid, o-phenylphenol, N-methylmorpholine-acetonitrile (MMA), 2-benzyl-4-chlorophenol, 2,2′-methylene-bis-(6-bromo-4-chlorophenol), 4,4′-dichloro-2′-hydroxydiphenyl ether (dichlosan), 2,4,4′-trichloro-2′-hydroxydiphenyl ether (trichlosan), chlorhexidine, N-(4-chlorophenyl)-N-(3,4-dichlorophenyl)-urea, N,N′-(1,10-decanediyldi-1-pyridinyl-4-ylidene)-bis-(1-octamine) dihydrochloride, N,N′bis-(4-chlorophenyl)-3,12-diimino-2,4,11,13-tetraaza-tetradecanediimideamide, glucoprotamines, surface-active antimicrobial quaternary compounds, guanidines, including the bi- and polyguanidines, such as for example 1,6-bis(2-ethylhexylbiguanidohexane) dihydrochloride, 1,6-di-(N₁,N₁′-phenyldiguanido-N₅,N₅′)hexane tetrahydrochloride, 1,6-di-(N₁,N₁-phenyl-N₁,N₁-methyldiguanido-N₅,N₅′)hexane dihydrochloride, 1,6-di-(N₁,N₁′-o-chlorophenyldiguanido-N₅,N₅′)hexane dihydrochloride, 1,6-di-(N₁,N₁′-2,6-dichlorophenyldiguanido-N₅,N₅′)hexane dihydrochloride, 1,6-di-[N₁,N₁′-β-(p-methoxyphenyl) diguanido-N₅,N₅′]hexane dihydrochloride, 1,6-di-(N₁,N₁′-α-methyl-β-phenyldiguanido-N₅,N₅′)hexane dihydrochloride, 1,6-di-(N₁,N ₁′-p-nitrophenyldiguanido-N₅,N₅′)hexane dihydrochloride, ω:ω-di-(N₁,N₁′-phenyldiguanido-N₅,N₅′)di-n-propyl ether dihydrochloride, ω:ω-di-(N₁,N_{1′}-p-chlorophenyldiguanido-N₅,N₅′)di-n-propyl ether tetrahydrochloride, 1,6-di-(N₁,N₁′-2,4-dichlorophenyldiguanido-N₅,N₅′)hexane tetrahydrochloride, 1,6-di-(N₁,N₁′-p-methylphenyldiguanido-N₅,N₅′)hexane dihydrochloride, 1,6-di-(N₁,N₁′-2,4,5-trichlorophenyldiguanido-N₅,N₅′)hexane tetrahydrochloride, 1,6-di-[N₁,N₁′-α-(p-chlorophenyl)ethyldiguanido-N₅,N₅′]hexane dihydrochloride, ω:ω-di-(N₁,N₁′-p-chlorophenyldiguanido-N₅,N_{5′})ₘ-xylene dihydrochloride, 1,12-di-(N₁,N₁′-p-chlorophenyldiguanido-N₅,N₅′)dodecane dihydrochloride, 1,10-di-(N₁,N₁′-phenyldiguanido-N₅,N₅′)decane tetrahydrochloride, 1,12-di-(N₁,N₁′-phenyldiguanido-N₅,N₅′)dodecane tetrahydrochloride, 1,6-di-(N₁,N₁′-o-chlorophenyldiguanido-N₅,N₅′)hexane dihydrochloride, 1,6-di-(N₁,N₁′-o-chlorophenyldiguanido-N₅,N₅′)hexane tetrahydrochloride, ethylene-bis-(1-tolylphenylbiguanide), ethylene-bis-(p-tolylphenylbiguanide), ethylene-bis-(3,5-dimethylphenylbiguanide), ethylene-bis-(p-tert-amylphenylbiguanide), ethylene-bis-(nonylphenylbiguanide), ethylene-bis-(phenylbiguanide), ethylene-bis-(N-butylphenylbiguanide), ethylene-bis-(2,5-diethoxyphenylbiguanide), ethylene-bis-(2,4-dimethylphenylbiguanide), ethylene-bis-(o-diphenylbiguanide), ethylene-bis-(mixed amylnaphthylbiguanide), N-butylethylene-bis-(phenylbiguanide), trimethylene bis(o-tolylbiguanide), N-butyltrimethylene-bis-(phenylbiguanide) and the corresponding salts like acetates, gluconates, hydrochlorides, hydrobromides, citrates, bisulfites, fluorides, polymaleates, N-coco alkyl sarcinosates, phosphites, hypophosphites, perfluorooctanoates, silicates, sorbates, salicylates, maleates, tartrates, fumarates, ethylenediaminetetraacetates, iminodiacetates, cinnamates, thiocyanates, arginates, pyromellitates, tetracarboxybutyrates, benzoates, glutarates, monofluorophosphates, perfluoropropionates as well as any mixtures thereof. Furthermore, halogenated xylene- and cresol derivatives are suitable, such as p-chloro-meta-cresol, p-chloro-meta-xylene, as well as natural antimicrobial active agents of plant origin (e.g. from spices or aromatics), animal as well as microbial origin. Preferred antimicrobial agents are antimicrobial surface-active quaternary compounds, a natural antimicrobial agent of vegetal origin and/or a natural antimicrobial agent of animal origin and, most preferably, at least one natural antimicrobial agent of vegetal origin from the group comprising caffeine, theobromine and theophylline and essential oils, such as eugenol, thymol and geraniol, and/or at least one natural antimicrobial agent of animal origin from the group comprising enzymes, such as protein from milk, lysozyme and lactoperoxidase and/or at least one antimicrobial surface-active quaternary compound containing an ammonium, sulfonium, phosphonium, iodonium or arsonium group, peroxy compounds and chlorine compounds. Substances of microbial origin, so-called bacteriozines, may also be used.

The quaternary ammonium compounds (QUATS) suitable as antimicrobial agents have the general formula (R¹)(R²)(R³)(R⁴)N⁺X⁻, in which R¹ to R⁴ may be the same or different and represent C₁₋₂₂ alkyl groups, C₇₋₂₈ aralkyl groups or heterocyclic groups, two or—in the case of an aromatic compound, such as pyridine—even three groups together with the nitrogen atom forming the heterocycle, for example a pyridinium or imidazolinium compound, and X⁻ represents halide ions, sulfate ions, hydroxide ions or similar anions. In the interests of optimal antimicrobial activity, at least one of the substituents preferably has a chain length of 8 to 18 and, more preferably, 12 to 16 carbon atoms.

QUATS can be obtained by reacting tertiary amines with alkylating agents such as, for example, methyl chloride, benzyl chloride, dimethyl sulfate, dodecyl bromide and also ethylene oxide. The alkylation of tertiary amines having one long alkyl chain and two methyl groups is particularly easy. The quaternization of tertiary amines containing two long chains and one methyl group can also be carried out under mild conditions using methyl chloride. Amines containing three long alkyl chains or hydroxy-substituted alkyl chains lack reactivity and are preferably quaternized with dimethyl sulfate.

Suitable QUATS are, for example, benzalkonium chloride (N-alkyl-N,N-dimethylbenzyl ammonium chloride, CAS No. 8001-54-5), benzalkon B (m,p-dichlorobenzyl dimethyl-C₁₂-alkyl ammonium chloride, CAS No. 58390-78-6), benzoxonium chloride (benzyldodecyl-bis-(2-hydroxyethyl) ammonium chloride), cetrimonium bromide (N-hexadecyl-N,N-trimethyl ammonium bromide, CAS No. 57-09-0), benzetonium chloride (N,N-di-methyl-N-[2-[2-[p-(1,1,3,3-tetramethylbutyl)-phenoxy]-ethoxy]-ethyl]-benzyl ammonium chloride, CAS No. 121-54-0), dialkyl dimethyl ammonium chlorides, such as di-n-decyldimethyl ammonium chloride (CAS No. 7173-51-5-5), didecyldimethyl ammonium bromide (CAS No. 2390-68-3), dioctyl dimethyl ammonium chloride, 1-cetylpyridinium chloride (CAS No. 123-03-5) and thiazoline iodide (CAS No. 1576448-1) and mixtures thereof. Particularly preferred QUATS are the benzalkonium chlorides containing C₈₋₁₈ alkyl groups, more particularly C₁₂₋₁₄ alkyl benzyl dimethyl ammonium chloride.

Benzalkonium halides and/or substituted benzalkonium halides are commercially obtainable, for example, as Barquat® from Lonza, Marquato® from Mason, Variquat® from Witco/Sherex and Hyamine® from Lonza and as Bardac® from Lonza. Other commercially obtainable antimicrobial agents are N-(3-chloroallyl)-hexaminium chloride, such as Dowicide® and Dowicil® from Dow, benzethonium chloride, such as Hyamine® 1622 from Rohm & Haas, methyl benzethonium chloride, such as Hyamine® 10X from Rohm & Haas, cetyl pyridinium chloride, such as cepacolchloride from Merrell Labs.

The antimicrobial agents are used in quantities of 0.0001% by weight to 1% by weight, preferably 0.001% by weight to 0.8% by weight, particularly preferably 0.005% by weight to 0.3% by weight and most preferably 0.01 to 0.2% by weight.

The agents may also comprise UV absorbers, which attach to the treated textiles and improve the light stability of the fibers and/or the light stability of the various ingredients of the formulation. UV-absorbers are understood to mean organic compounds, which are able to absorb UV radiation and emit the resulting energy in the form of longer wavelength radiation, for example as heat. Compounds, which possess these desired properties, are for example, the efficient radiationless deactivating derivatives of benzophenone having substituents in position(s) 2 and/or 4. Also suitable are substituted benzotriazoles, acrylates, which are phenyl-substituted in position 3 (cinnamic acid derivatives), with or without cyano groups in position 2, salicylates, organic Ni complexes, as well as natural substances such as umbelliferone and the endogenous urocanic acid. The biphenyl and above all the stilbene derivatives such as for example those described in EP 0728749 A and commercially available as Tinosorb® FD or Tinosorb® FR from Ciba, are of particular importance. As UV-B absorbers can be cited: 3-benzylidenecamphor or 3-benzylidenenorcamphor and its derivatives, for example 3-(4-methylbenzylidene) camphor, as described in the EP 0693471 B1; 4-aminobenzoic acid derivatives, preferably 4-(dimethylamino)benzoic acid, 2-ethylhexyl ester, 4(dimethylamino)benzoic acid, 2-octyl ester and 4-(dimethylamino)benzoic acid, amylester; esters of cinnamic acid, preferably 4-methoxycinnamic acid, 2-ethylhexyl ester, 4-methoxycinnamic acid, propyl ester, 4-methoxycinnamic acid, isoamyl ester, 2-cyano-3,3-phenylcinnamic acid, 2-ethylhexyl ester (octocrylene); esters of salicylic acid, preferably salicylic acid, 2-ethylhexyl ester, salicylic acid, 4-isopropylbenzyl ester, salicylic acid, homomenthylester; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably 4-methoxybenzmalonic acid, di-2-ethylhexylester; triazine derivatives, such as, for example 2,4,6-trianilino-(p-carbo-2′-ethyl-1′-hexyloxy)-1,3,5-triazine and octyl triazone, as described in EP 0818450 A1 or dioctyl butamidotriazone (Uvasorb® HEB); propane-1,3-dione, such as for example 1-(4-tert. butylphenyl)-3-(4′-methoxyphenyl)propane-1,3-dione; ketotricyclo(5.2.1.0) decane derivatives, as described in EP 0694521 B1. Further suitable are 2-phenylbenzimidazole-5-sulfonic acid and its alkali-, earth alkali-, ammonium-, alkylammonium-, alkanolammonium- and glucammonium salts; sulfonic acid derivatives of benzophenones, preferably 2-hydroxy-4-methoxybenzophenone-5-sulfonic acid and its salts; sulfonic acid derivatives of 3-benzylidenecamphor, as for example 4-(2-oxo-3-bornylidenemethyl)benzene sulfonic acid and 2-methyl-5-(2-oxo-3-bornylidene) sulfonic acid and its salts.

Typical UV-A filters particularly include derivatives of benzoylmethane, such as, for example 1-(4′-tert.-butylphenyl)-3-(4′-methoxyphenyl)propane-1,3-dione, 4-tert.-butyl-4′-methoxydibenzoylmethane (Parsol 1789), 1-phenyl-3-(4′-isopropylphenyl)-propane-1,3-dione as well as enamine compounds, as described in the DE 19712033 A1 (BASF). Naturally, the UV-A and UV-B filters can also be added as mixtures. Beside the cited soluble materials, also insoluble, light protective pigments, namely finely dispersed, preferably, nano metal oxides or salts can be considered for this task. Exemplary suitable metal oxides are particularly zinc oxide and titanium oxide and also oxides of iron, zirconium, silicon, manganese, aluminum and cerium as well as their mixtures. Silicates (talk), barium sulfate or zinc stearate cab be added as salts. The oxides and salts are already used in the form of pigments for skin care and skin protecting emulsions and decorative cosmetics. Here, the particles should have an average diameter of less than 100 nm, preferably between 5 and 50 nm and especially between 15 and 30 nm. They can be spherical, however elliptical or other shaped particles can be used. The pigments can also be surface treated, i.e. hydrophilized or hydrophobized. Typical examples are coated titanium dioxides, such as, for example Titandioxid Z 805 (Degussa) or Eusolex® T2000 (Merck); hydrophobic coating agents preferably include trialkoxy octylsilanes or silicones. Micronized zinc oxide is preferably used. Further suitable UV light protection filters may be found in the review by P. Finkel in SöFW-Journal, volume 122 (1996), p. 543.

The w absorbers are normally used in amounts of 0.01 wt. % to 5 wt. %, preferably from 0.03 wt. % to 1 wt. %.

To increase their washing or cleaning power, agents according to the invention can comprise, in addition to the enzymes according to the invention, additional enzymes, in principle any enzyme established for these purposes in the prior art being useable. These particularly include proteases, amylases, lipases, hemicellulases, cellulases or oxidoreductases as well as preferably their mixtures. In principle, these enzymes are of natural origin; improved variants based on the natural molecules are available for use in detergents and accordingly they are preferred. The detergents according to the invention preferably comprise enzymes in total quantities of 1×10⁻⁶ to 5 weight percent based on active protein. Protein concentrations can be determined using known methods, for example the BCA Process (bicinchoninic acid; 2,2′-bichinolyl-4,4′-dicarboxylic acid) or the biuret process (A. G. Gomall, C. S. Bardawill and M. M. David, J. Biol. Chem., 177 (1948), p. 751-766).

Preferred proteases are those of the subtilisin type. Examples of these are subtilisins BPN′ and Carlsberg, the protease PB92, the subtilisins 147 and 309, the alkaline protease from Bacillus lentus, subtilisin DY and those enzymes of the subtilases no longer however classified in the stricter sense as subtilisins thermitase, proteinase K and the proteases TW3 und TW7. Subtilisin Carlsberg in further developed form is available under the trade name Alcalase® from Novozymes A/S, Bagsvaerd, Denmark. Subtilisins 147 and 309 are commercialized under the trade names Esperase® and Savinase® by the Novozymes company. Variants derived from the protease from Bacillus lentus DSM 5483 (WO 91/02792 A1) are called BLAP®, described particularly in WO 92/21760 A1, WO 95/23221 A1, WO 02/088340 A2 and WO 03/038082 A2. Further suitable proteases from various Bacillus sp. and B. gibsonii emerge from the Patent applications WO 03/054185 A1, WO 03/056017 A2, WO 03/055974 A2 and WO 03/054184 A1.

Further useable proteases are, for example, those enzymes available with the trade names Durazym®, Relase®, Everlase®, Nafizym, Natalase®, Kannase® and Ovozymes® from the Novozymes Company, those under the trade names Purafect®, Purafect® OxP and Properase® from Genencor, that under the trade name Protosol® from Advanced Biochemicals Ltd., Thane, India, that under the trade name Wuxi® from Wuxi Snyder Bioproducts Ltd., China, those under the trade names Proleather® and Protease P® from Amano Pharmaceuticals Ltd., Nagoya, Japan, and that under the designation Proteinase K-16 from Kao Corp., Tokyo, Japan.

Examples of further useable amylases according to the invention are the α-amylases from Bacillus licheniformis, from B. amyloliquefaciens and from B. stearothermophilus, as well as their improved further developments for use in detergents. The enzyme from B. licheniformis is available from the Company Novozymes under the name Termamyl® and from the Genencor Company under the name Purastar®ST. Further development products of this α-amylase are available from the Company Novozymes under the trade names Duramyl® and Termamyl®ultra, from the Company Genencor under the name Purastar®OxAm and from the Company Daiwa Seiko Inc., Tokyo, Japan as Keistase®. The α-amylase from B. amyloliquefaciens is commercialized by the Company Novozymes under the name BAN®, and derived variants from the α-amylase from B. stearothermophilus under the names BSG® and Novamyl®, also from the Company Novozymes.

Moreover, for these purposes, attention should be drawn to the α-amylase from Bacillus sp. A 7-7 (DSM 12368) disclosed in the application WO 02/10356 A2 and the cyclodextrin-glucanotransferase (CGTase) from B. agaradherens (DSM 9948) described in WO 02/44350 A2. Amylolytic enzymes can also be used, which belong to the sequence space of α-amylases, defined in the application WO 03/002711 A2, and those described in the application WO 03/054177 A2. Similarly the fusion products of the cited molecules can be used, for example those in the application DE 10138753 A1.

Moreover, further developments of α-amylase from Aspergillus niger und A. oryzae available from the Company Novozymes under the trade name Fungamyl® are suitable. A further suitable commercial product is, for example Amylase-LT®.

The agents according to the invention can comprise lipases or cutinases, particularly due to their triglyceride cleaving activities, but also in order to produce in situ peracids from suitable preliminary steps. These include the available or further developed lipases originating from Humicola lanuginosa (Thermomyces lanuginosus), particularly those with the amino acid substitution D96L. They are commercialized, for example by the Novozymes Company under the trade names Lipolase®, Lipolase®Ultra, LipoPrime®, Lipozyme® and Lipex®. Moreover, suitable cutinases, for example are those that were originally isolated from Fusarium solani pisi and Humicola insolens. Likewise useable lipases are available from the Amano Company under the designations Lipase CE®, Lipase P®, Lipase B®, and Lipase CES®, Lipase AKG®, Bacillis sp. Lipase®, Lipase AP®, Lipase M-AP® and Lipase AML®. Suitable lipases or cutinases whose starting enzymes were originally isolated from Pseudomonas mendocina und Fusarium solanii are for example available from Genencor Company. Further important commercial products that may be mentioned are the commercial preparations M1 Lipase® und Lipomax® originally from Gist-Brocades Company, and the commercial enzymes from the Meito Sangyo KK Company, Japan under the names Lipase MY-30®, Lipase OF® and Lipase PL® as well as the product Lumafast® from Genencor Company.

Agents according to the invention, particularly when they are destined for treating textiles, can comprise cellulases, according to their purpose, as pure enzymes, as enzyme preparations, or in the form of mixtures, in which the individual components advantageously complement their various performances. Among these aspects of performance are particular contributions to primary washing performance, to secondary washing performance of the product, (anti-redeposition activity or inhibition of graying) and softening or brightening (effect on the textile), through to performing a “stone washed” effect.

A usable, fungal endoglucanase(EG)-rich cellulase preparation, or its further developments are offered by the Novozymes Company under the trade name Celluzyme®. The products Endolase® and Carezyme® based on the 50 kD-EG, respectively 43 kD-EG from H. insolens DSM 1800 are also obtainable from Novozymes Company. Further commercial products from this company are Cellusoft® and Renozyme®, based on the application WO 98/12307 A1. Cellulase variants with improved performance emerge from the application WO 98/12307 A1. Similarly, the cellulases disclosed in application WO 97/14804 A1 can be used; for example 20 kD-EG cellulase from Melanocarpus, obtainable from AB Enzymes Company, Finland under the trade names Ecostone® and Biotouch®. Further commercial products from the AB Enzymes Company are Econase® and Ecopulp®. A further suitable cellulase from Bacillus sp. CBS 670.93 is obtainable from the Genencor Company under the trade name Puradax®. Additional commercial products from the Genencor Company are “Genencor detergent cellulase L” and Indiage®Neutra.

Particularly for removing specific problematic stains, the agents according to the invention can comprise additional enzymes, which are summarized under the term hemicellulases. These include, for example mannanases, xanthanlyases, pectinlyases (=pectinases), pectinesterases, pectatlyases, xyloglucanases (=xylanases), pullulanases and β-glucanases. Suitable mannanases, for example are available under the names Gamanase® and Pektinex AR® from Novozymes Company, under the names Rohapec® B1L from AB Enzymes and under the names Pyrolase® from Diversa Corp., San Diego, Calif., USA. A suitable β-Glucanase from a B. alcalophilus emerges from the application WO 99/06573 A1, for example. β-Glucanase extracted from B. subtilis is available under the name Cereflo® from Novozymes Company.

To increase the bleaching action, the washing or cleaning agents can comprise oxidoreductases, for example oxidases, oxygenases, katalases, peroxidases, like halo-, chloro-, bromo-, lignin-, glucose- or manganese-peroxidases, dioxygenases or laccases (phenoloxidases, polyphenoloxidases). Suitable commercial products are Denilite® 1 and 2 from the Novozymes Company. Advantageously, additional, preferably organic, particularly preferably aromatic compounds are added that interact with the enzymes to enhance the activity of the relative oxidoreductases (enhancers) or to facilitate the electron flow (mediators) between the oxidizing enzymes and the stains over strongly different redox potentials.

The enzymes used in the agents according to the invention either stem originally from microorganisms, such as the species Bacillus, Streptomyces, Humicola, or Pseudomonas, and/or are produced according to known biotechnological processes using suitable microorganisms such as by transgenic expression hosts of the species Bacillus or filamentary fungi.

Purification of the relevant enzymes follows conveniently using established processes such as precipitation, sedimentation, concentration, filtration of the liquid phases, microfiltration, ultrafiltration, mixing with chemicals, deodorization or suitable combinations of these steps.

The enzymes can be added to the agents according to the invention in each established form according to the prior art. Included here, for example, are solid preparations obtained by granulation, extrusion or lyophilization, or particularly for liquid agents or agents in the form of gels, enzyme solutions, advantageously highly concentrated, of low moisture content and/or mixed with stabilizers.

Alternatively, all enzymes, both for solid as well as for liquid presentation forms, can be encapsulated, for example by spray drying or extrusion of the enzyme solution together with a preferably natural polymer or in the form of capsules, for example those in which the enzyme is embedded in a solidified gel, or in those of the core-shell type, in which an enzyme-containing core is covered with a water-, air- and/or chemical-impervious protective layer. Further active principles, for example stabilizers, emulsifiers, pigments, bleaches or colorants can be applied in additional layers. Such capsules are made using known methods, for example by vibratory granulation or roll compaction or by fluid bed processes. Advantageously, these types of granulates, for example with an applied polymeric film former are dust-free and as a result of the coating are storage stable.

In addition, it is possible to formulate two or more enzymes together, so that a single granule exhibits a plurality of enzymatic activities.

Protein concentrations on the comprised enzymes, particularly on the comprised choline oxidases can be determined using known methods, for example the BCA Process (bicinchoninic acid; 2,2′-bichinolyl-4,4′-dicarboxylic acid) or the biuret process (A. G. Gornall, C. S. Bardawill and M. M. David, J. Biol. Chem., 177 (1948), p. 751-766).

A protein and/or enzyme in an agent according to the invention can be protected, particularly in storage, against deterioration such as, for example inactivation, denaturation or decomposition, for example through physical influences, oxidation or proteolytic cleavage. An inhibition of the proteolysis is particularly preferred during microbial. preparation of proteins and/or enzymes, particularly when the agents also contain proteases. According to the invention, stabilizers can be added for this purpose.

One group of stabilizers are reversible protease inhibitors. For this, benzamidine hydrochloride, borax, boric acids, boronic acids or their salts or esters are frequently used, above all derivatives with aromatic groups, for example ortho, meta or para substituted phenyl boronic acids, or their salts or esters. Peptide aldehydes, i.e. oligopeptides with a reduced C-terminus, are also suitable. Ovomucoid and leupeptin, among others, belong to the peptidic reversible protease inhibitors; an additional option is the formation of fusion proteins from proteases and peptide inhibitors.

Further enzyme stabilizers are amino alcohols like mono-, di-, triethanol- and -propanolamine and their mixtures, aliphatic carboxylic acids up to C₁₂, such as for example succinic acid, other dicarboxylic acids or salts of the cited acids. End-capped fatty acid amide alkoxylates are also suitable stabilizers. Specific organic acids, added as builders, are in addition capable of stabilizing a comprised enzyme, as disclosed in WO 97/18287.

Lower aliphatic alcohols, but above all polyols such as, for example glycerol, ethylene glycol, propylene glycol or sorbitol are further frequently used enzyme stabilizers. Di-glycerol phosphate also protects against denaturation by physical influences. Similarly, calcium and/or magnesium salts are used, such as, for example calcium acetate or calcium formate.

Polyamide oligomers or polymeric compounds like lignin, water-soluble vinyl copolymers or cellulose ethers, acrylic polymers and/or polyamides stabilize enzyme preparations against physical influences or pH variations. Polymers containing polyamine-N-oxide act simultaneously as enzyme stabilizers and color transfer inhibitors. Other polymeric stabilizers are linear C₈-C₁₈ polyoxyalkylenes. Alkyl polyglycosides can also stabilize the enzymatic components of the agents according to the invention and in addition, induce them to increase in performance. Crosslinked nitrogen-containing compounds perform a dual function as soil release agents and as enzyme stabilizers. Hydrophobic, nonionic polymer stabilizes in particular, an optionally comprised cellulase.

Reducing agents and antioxidants increase the stability of enzymes against oxidative decomposition; examples of these are such as sulfur-containing reducing agents. Other examples are sodium sulfite and reducing sugar.

The use of combinations of stabilizers is particularly preferred, for example of polyols, boric acid and/or borax, the combination of boric acid or borate, reducing salts and succinic acid or other dicarboxylic acids or the combination of boric acid or borate with polyols or polyamino compounds and with reducing salts. The effect of peptide-aldehyde stabilizers is conveniently increased by the combination with boric acid and/or boric acid derivatives and polyols and still more by the additional effect of divalent cations, such as for example calcium ions.

In a preferred embodiment, agents according to the invention are characterized in that they consist of more than one phase in order to liberate the comprised active principles separately from one another at different times or from different places, for example. This can concern phases in different aggregates, however it particularly concerns phases in the same aggregates.

Agents according to the invention, which are composed of more than one solid component, can be easily manufactured by mixing together the various solid components in bulk form, particularly powders, granules or extrudates with various ingredients and/or different release behavior. The manufacture of solid agents with one or more phases according to the invention can be made by known methods, for example by spray drying or granulation, wherein the enzymes and possible further heat-sensitive ingredients, such as, for example bleaches are optionally added separately. For manufacturing agents according to the invention with an increased bulk density, particularly in the range of 650 g/l to 950 g/l, a preferred process is one with an extrusion step, known from the European Patent EP 0 486 592. A further preferred manufacturing using a granulation process is described in the European Patent EP 0 642 576.

For solids, proteins can be used, for example, in dried, granulated, encapsulated or encapsulated and additionally dried form. They can be added separately, i.e. as one phase, or together with other ingredients in the same phase, with or without compaction. If microencapsulated, solid enzymes are used, then the water can be removed from the aqueous solutions resulting from the process by means of processes known from the prior art, such as spray-drying, centrifugation or by transdissolution. The particles obtained in this manner normally have a particle size between 50 and 200 μm.

The encapsulated form also serves, as previously discussed, to protect the enzymes from other ingredients such as bleaches, or to enable a controlled release. These capsules are differentiated by size as millicapsules, microcapsules and nanocapsules; microcapsules being particularly preferred for enzymes. Such capsules are disclosed, for example, in the Patent applications WO 97/24177 and DE 199 18 267. Another possible encapsulation method consists in the encapsulation of the enzymes suitable for washing or cleaning agents in starch or in starch derivatives, starting from a mixture of the enzyme solutions with a solution or suspension of starch or a starch derivative. Such an encapsulation process is described in the German application DE 199 56 382.

At least two solid phases can also be combined with each other. Thus, it is possible to prepare a solid agent according to the invention, by compression or compaction into tablets. Such tablets can be monophase or multiphase tablets. Consequently, this presentation form also offers the possibility of displaying a solid agent having two solid phases according to the invention. For manufacturing the agents in tablet form according to the invention, which can be monophasic or multiphasic, single colored or multicolored and/or consisting of one or several layers, all the ingredients—optionally for each layer—are preferably mixed together in a mixer and the mixture is compressed using conventional tablet presses, e.g. exocentric presses or rotating presses with compression forces in the range of about 50 to 100 kN/cm², preferably 60 to 70 kN/cm². Particularly for the case of multilayer tablets, it can be advantageous to precompress at least one layer. This is preferably carried out using compression forces between 5 and 20 kN/cm², particularly 10 to 15 kN/cm². Tablets prepared in this way preferably have a weight of 10 g to 50 g, particularly 15 g to 40 g. The tablets may be any shape—round, oval or angled—intermediate shapes also being possible.

It is particularly advantageous for multiphase agents, that at least one of the phases comprises an amylase-sensitive material, especially starch, or is at least partially encapsulated or coated with this. In this way this phase is mechanically stabilized and/or protected against external influences and simultaneously attacked by an active amylase present in the wash liquor, such that the release of the ingredients is facilitated.

Similarly, preferred agents according to the invention are characterized in that they are all in liquid, gel or paste form. The proteins, preferably a protein according to the invention, are added to such agents and preferably result from a state of the art protein extraction and preparation in concentrated aqueous or non-aqueous solution, for example in liquid form, such as solution, suspension or emulsion, but also in gel form or encapsulated or as dried powder. This type of washing or cleaning agent according to the invention in the form of solutions in standard solvents are generally prepared by a simple mixing of the ingredients, which can be added in the substance or as a solution into an automatic mixer.

An embodiment of the present invention is such a liquid, gel or paste agent, to which has been added an encapsulated protein essential for the invention and/or one of the other comprised proteins and/or one of the other comprised ingredients in the form of microcapsules. Among these, those encapsulated with amylase-sensitive materials are particularly preferred. The use of a combination of amylase-sensitive materials and an amylolytic enzyme in a washing or cleaning agent can demonstrate synergistic effects in such a way that the starch degrading enzyme supports the breakdown of the microcapsule and thereby controls the release process of the encapsulated ingredients with the result that the release does not happen during storage and/or not at the beginning of the cleaning process, but rather at a defined time. By this mechanism, complex washing and cleaning agent systems can be based on the most varied ingredients and the most varied capsule types, which represent the particularly preferred embodiments of the present invention.

A comparable effect is given when the ingredients of the washing and cleaning agent are distributed in at least two different phases, for example two or more solid associated phases of a tableted washing or cleaning agent, or different granules in the same powdery agent. Two-phase or multi-phase cleaners are state of the art for use in both automatic dishwashers as well as washing agents. The activity of an amylolytic enzyme in an earlier activated phase is a prerequisite for the activation of a later phase, when this is surrounded by an amylase-sensitive shell or coating, or the amylase-sensitive material represents an integral part of the solid phase, whose partial or total hydrolysis disintegrates the relevant phase.

The ingredients of washing and cleaning agents are able to suitably support each other's performance. Thus, it is known from the application WO 98/45396, that polymers, which can be added as cobuilders, such as, for example alkyl polyglycosides, can simultaneously stabilize and augment the activity and stability of included enzymes. Accordingly, it is preferred when a Carlsberg variant according to the invention is modified by one of the customary ingredients mentioned above, especially stabilized and/or its contribution to the performance of the washing or cleaning agent is increased.

Processes for cleaning textiles or hard surfaces constitute a further subject of the invention and are characterized in that an above-described choline oxidase variant according to the invention is active in at least one of the process steps.

In this embodiment, the invention is realized in that the improved enzymatic properties according to the invention are utilized in principal in terms of an improvement in each cleaning process. Each cleaning process is enhanced by the relevant activity when it is present in each process step. Such processes are realized in machines such as standard household automatic dishwashers or household washing machines. Further preferred processes are those wherein the choline oxidase variants are added in an above-described agent.

A further subject of the invention is a shampoo and/or a hair care agent comprising choline oxidases useable according to the invention and particularly choline oxidases according to the invention.

The shampoos and/or hair care products as well as bubble baths, shower baths, creams, gels, lotions, alcoholic and aqueous-alcoholic solutions, emulsions, wax/fatty masses, sticks, powder or salves that include choline oxidases useable according to the invention and particularly choline oxidases according to the invention can contain mild surfactants, oils, emulsifiers, greases, pearlescent waxes, consistence providers, thickeners, polymers, silicone compounds, fats, waxes, stabilizers, biogenetic active principles, deodorants, antiperspirants, anti-dandruff agents, film formers, swelling agents, UV-light protection factors, antioxidants, hydrotropes, conserving agents, insect repellants, sun tans, solubilizers, perfume oils, colorants and the like as auxiliaries and additives.

Typical examples of suitable mild, i.e. particularly skin-compatible surfactants are fatty alcohol polyglycol ether sulfonates, monoglyceride sulfates, mono and/or dialkylsulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines and/or protein-fatty acid condensates, the last preferably on the basis of wheat proteins.

The following can be considered as oils, for example: Guerbet alcohols based on fatty alcohols with 6 to 18, preferably 8 to 10 carbon atoms, esters of linear C₆-C₂₂-fatty acids with linear C₆-C₂₂-fatty alcohols, esters of branched C₆-C₁₃- carboxylic acids with linear C₆-C₂₂-fatty alcohols, such as for example myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. In addition, suitable esters are esters of linear C₆-C₂₂-fatty acids with branched alcohols, especially 2-ethylhexanol, esters of hydroxycarboxylic acids with linear or branched C₆-C₂₂-fatty alcohols, especially dioctyl malate, esters of linear and/or branched fatty acids with polyhydroxy alcohols (e.g. propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₁₈-fatty alcohols and/or Guerbet alcohols with aromatic carboxylic acids, especially benzoic acid, esters of C₂-C₂₂-dicarboxylic acids with linear or branched alcohols with 1 to 22 carbon atoms or polyols with 2 to 10 carbon atoms and 2 to 6 hydroxyl groups, vegetal oils, branched primary alcohols, substituted cyclohexanes, linear and branched C₆-C₂₂-fatty alcohol carbonates, Guerbet carbonates, esters of benzoic acid with linear and/or branched C₆-C₂₂-alkohols (e.g. Finsolv® TN), linear or branched, symmetrical or unsymmetrical dialkyl ethers with 6 to 22 carbon atoms per alkyl group, ring opening products of epoxidized fatty acid esters with polyols, silicone oils and/or aliphatic or naphthenic hydrocarbons, such as, for example squalane, squalene or dialkylcyclohexanes.

Emulsifiers can be selected for example from nonionic surfactants from at least one of the following groups:
Addition products of 2 to 30 moles ethylene oxide and/or 0 to 5 moles propylene oxide to fatty alcohols with 8 to 22 carbon atoms, to fatty acids with 12 to 22 carbon atoms, to alkyl phenols with 8 to 15 carbon atoms in the alkyl group as well as alkylamines with 8 to 22 carbon atoms in the alkyl radical;
C_{12/16}-fatty acid mono and diesters of addition products of 1 to 30 moles ethylene oxide on glycerin;
Glycerin mono and diesters and sorbitol mono and diesters of saturated and unsaturated fatty acids with 6 to 22 carbon atoms and their ethylene oxide addition products;
Alkyl- and/or alkenyl mono and -oligoglycosides with 8 to 22 carbon atoms in the alk(en)yl radical and their ethoxylated analogs;
Addition products of 2 to 15 moles ethylene oxide on castor oil and/or hydrogenated castor oil;
Polyol esters and especially polyglycerin esters;
Addition products of 2 to 15 moles ethylene oxide on castor oil and/or hydrogenated castor oil;
Partial esters based on linear, branched, unsaturated or saturated C₆/22-fatty acids, ricinoleic acid as well as 12-hydroxystearic acid and glycerin, polyglycerin, pentaerythritol, dipentaerythritol, sugar alcohols (e.g. sorbitol), alkyl glucosides (e.g. methyl glucoside, butyl glucoside, lauryl glucoside) as well as polyglucosides (e.g. cellulose);
Mono, di and trialkyl phosphates as well as mono, di and/or tri-PEG-alkylphosphates and salts thereof;
Wool wax alcohols;
Polysiloxane-polyalkyl-polyether copolymers or corresponding derivatives;
Mixed esters of pentaerythritol, fatty acids, citric acid and fatty alcohol according to the Patent DE 1165574 and/or mixed esters of fatty acids with 6 to 22 carbon atoms, methyl glucose and polyols, preferably glycerin or polyglycerin,
Polyalkylene glycols and
Glycerin carbonate.

The addition products of ethylene oxide and/or propylene oxide on fatty alcohols, fatty acids, alkyl phenols, glycerin mono and diesters as well as sorbitol mono and diesters of fatty acids or on castor oil represent known, commercially available products. They can be considered as mixtures of homologs, whose mean degree of alkoxylation corresponds to the ratio of amounts of ethylene oxide and/or propylene oxide, used for the addition reaction, and that of the substrate. C_{12/18} fatty acid mono and diesters of addition products of ethylene oxide on glycerin are known from DE 2024051 as greasing agents for cosmetic preparations.

Alkyl and/or alkenyl mono and oligoglycosides, their manufacture and use are known from the prior art. Their manufacture results particularly from the reaction of glucose or oligosaccharides with primary alcohols containing 8 to 18 carbon atoms. As far as the glycoside radicals are concerned, both monoglycosides, in which a cyclic sugar radical is glycosidically linked to the fatty alcohol, and also oligomeric glycosides, with a degree of oligomerization of preferably about 8, are suitable. In this context, the oligomerization degree is a statistical mean value based on the typical homolog distribution of such technical products.

Typical examples of suitable polyglycerin esters are polyglyceryl-2 dipolyhydroxystearate (Dehymuls® PGPH), polyglycerin-3-diisostearate (Lameform® TGI), polyglyceryl-4-isostearate (Isolan® GI 34), polyglyceryl-3 oleate, diisostearoyl polyglyceryl-3 diisostearate (Isolan® PDI), polyglyceryl-3 methylglucose distearate (Tego Care(D 450), polyglyceryl-3 beeswax (Cera Bellinag), polyglyceryl-4 caprate (polyglycerol caprate T2010/90), polyglyceryl-3 cetyl ether (Chimexane® NL), polyglyceryl-3 distearate (Cremophor® GS 32) and polyglyceryl polyricinoleate (Admul® WOL 1403) polyglyceryl dimerate isostearate and mixtures thereof.

Moreover, zwitterionic surfactants can be used as emulsifiers. Zwitterionic surfactants are designated as those surface-active compounds that carry at least a quaternary ammonium group and at least a carboxylate and a sulfonate group in the molecule. Particularly suitable zwitterionic surfactants are the so-called betaines such as the N-alkyl-N,N-dimethylammonium glycinates, for example the cocoalkyldimethylammonium glycinate, N-acylaminopropyl-N,N-dimethylammonium glycinates, for example the cocoacylaminopropyldimethylammonium glycinate, and 2-alkyl-3-carboxymethyl-3-hydroxyethyl imidazolines with 8 to 18 carbon atoms in each of the alkyl or acyl groups, as well as cocoacylaminoethylhydroxyethylcarboxymethyl glycinate. The known fatty acid derivative known under the CTFA-description Cocamidopropyl Betaine is particularly preferred. The ampholytic surfactants are understood to include such surface-active compounds that apart from a C_{8/18} alkyl or acyl group, contain at least one free amino group and at least one COOH or SO₃H group in the molecule, and are able to form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycine, N-alkyltaurines, N-alkylsarcosinea, 2-alkyl-aminopropionic acids und alkylaminoacetic acids with about 8 to 18 C-atoms in each alkyl group. Particularly preferred ampholytic surfactants are N-cocoalkylamino propionate, cocoacylaminoethylamino propionate and C_{12/18}-acyl sarcosine. Beside the ampholytics, the quaternary emulsifiers can also be considered, wherein the esterquats, preferably methylquaternized difatty acid triethanolamine ester salts are particularly preferred.

As greasing agents, substances such as lanolin and lecithin, as well as polyethoxylated or acylated lanolin and lecithin derivatives, polyol fatty acid esters, monoglycerides and fatty acid alkanolamides can be used, the last ones serving as foam stabilizers at the same time.

Pearlescent waxes include: alkylene glycol esters, especially ethylene glycol distearate; fatty acid alkanolamides, especially cocofatty acid diethanolamide; partial glycerides, especially monoglyceride of stearic acid; esters of polyfunctional, optionally hydroxy-substituted carboxylic acids with fatty alcohols with 6 to 22 carbon atoms, especially long chain esters of tartaric acid; solids, such as, for example fatty alcohols, fatty ketones, fatty aldehydes, fatty ethers and fatty carbonates, which have a total of at least 24 carbon atoms, especially lauron and distearyl ether; fatty acids like stearic acid, hydroxystearic acid or behenic acid, ring opened products of olefin epoxides having 12 to 22 carbon atoms with fatty alcohols with 12 to 22 carbon atoms and/or polyols having 2 to 15 carbon atoms and 2 to 10 hydroxyl groups and mixtures thereof.

Consistence agents primarily include fatty alcohols or hydroxyfatty alcohols having 12 to 22 and preferably 16 to 18 carbon atoms, besides partial glycerides, fatty acids or hydroxyfatty acids. A combination of these materials with alkyl oligoglucosides and/or fatty acid N-methylglucamides of the same chain length and/or polyglycerin poly-12-hydroxystearates is preferred.

Suitable thickeners are for example aerosil types (hydrophilic silicic acids), polysaccharides, especially xanthane gum, guar-guar, agar-agar, alginates and tyloses, carboxymethyl cellulose and hydroxyethyl cellulose, in addition, higher molecular polyethylene glycol mono- and-diesters of fatty acids, polyacrylates, (e.g. Carbopole® from Goodrich or Synthalene® from Sigma), polyacrylamides, Polyvinyl alcohol and polyvinyl pyrrolidone, surfactants such as ethoxylated fatty acid glycerides, esters of fatty acids with polyols such as pentaerythritol or trimethylolpropane, fatty alcohol lethoxylates with narrowed homolog distribution or alkyl oligoglucosides as well as electrolytes like cooking salt and ammonium chloride.

Exemplary suitable cationic polymers are cationic cellulose derivatives, such as a quaternized hydroxyethyl cellulose, available under the trade name Polymer JR 400® from Amerchol, cationic starches, copolymers of diallylammonium salts and acrylamides, quaternized vinyl pyrrolidone/vinyl imidazole polymers, like e.g. Luviquat® (BASF), condensation products of polyglycols with amines, quaternized collagen polypeptides, like for example, lauryldimonium hydroxypropyl hydrolyzed collagen (Lamequat®L/Grünau), quaternized wheat polypeptides, polyethylene imines, cationic silicone polymers, such as amidomethicone, copolymers of adipic acid and dimethylaminohydroxypropyldiethylene triamine (Cartaretine®/Sandoz), copolymers of acrylic acid and dimethyldiallylammonium chloride (Merquat® 550/Chemviron), polyaminopolyamides, such as e.g. described in FR 2252840 A as well as their crosslinked water-soluble polymers, cationic chitin derivatives such as e.g. quaternized chitosan, optionally microcystallinically dispersed, condensation products of dihaloalkylenes, such as e.g. dibromobutane with bisdialkylamines, such as e.g. bis-dimethylamino-1,3-propane, cationic guar gum, such as e.g. Jaguar® CBS, Jaguar® C-17, Jaguar® C-16 from Celanese company, quaternized ammonium polymers, such as e.g. Mirapol® A-1 5, Mirapol® AD-1, Mirapol® AZ-1 from the Miranol company.

Anionic, zwitterionic, amphoteric and nonionic polymers include, for example, vinyl acetate-crotonic acid copolymers, vinyl pyrrolidone-vinyl acrylate copolymers, vinyl acetate-butyl maleate-isobornyl acrylate copolymers, methyl vinyl ether-maleic anhydride copolymers and their esters, uncrosslinked polyacrylic acids and those crosslinked with polyols, acrylamidopropyltrimethylammonium chloride-acrylate copolymers, octylacylamide-methyl methacrylate-tert.-butylaminoethyl methacrylate-2-hydroxypropyl methacrylate copolymers, polyvinyl pyrrolidone, vinyl pyrrolidone-vinyl acetate copolymers, vinyl pyrrolidone-dimethylaminoethyl methacrylate-vinyl caprolactam terpolymers as well as optionally derivatized cellulose ethers and silicones.

Exemplary suitable silicone compounds are dimethylpolysiloxanes, methylphenylpolysiloxanes, cyclic siloxanes as well as amino-, fatty acid-, alcohol-, polyether-, epoxy-, fluorine-, glycoside- and/or alkyl modified silicone compounds, which may be both liquid or also resinous at room temperature. Simethicones, which are mixtures of dimethicones having an average chain length of 200 to 300 dimethylsiloxane units and hydrated silicates, are also suitable. A detailed review of suitable volatile silicones is found in Todd et al., Cosm. Toil. 91, 27 (1976).

Typical examples of fats are glycerides; waxes include inter alia natural waxes such as e.g. candelilla wax, carnauba wax, japan wax, esparto grass wax, cork wax, guarum wax, rice oilseed wax, raw sugar wax, ouricury wax, montan wax, beeswax, shellac wax, spermaceti, lanolin (wool wax), fowl fat, ceresine, ozokerite, petrolatum, paraffin waxes microwaxes; chemically modified waxes (hard waxes), such as e.g. montan ester waxes, sasol waxes, hydrogenated jojoba waxes as well as synthetic waxes, such as e.g. polyalkylene waxes and polyethylene glycol waxes.

Metal salts of fatty acids, such as e.g. magnesium-, aluminum- and/or zinc stearate or ricinoleate can be used as stabilizers.

Biogenetic active agents are understood to mean for example, tocopherol, tocopherol acetate, tocopherol palmitate, ascorbic acid, desoxyribonucleic acid, retinol, bisabolol, allantoin, phytanetriol, panthenol, AHA-acids, amino acids, ceramides, pseudoceramides, essential oils, plant extracts and vitamin complexes.

Cosmetic deodorants act against body odors, masking or eliminating them. Body odors result from the action of skin bacteria on apocrine sweat, whereby unpleasant smelling degradation products are formed. Accordingly, deodorants contain active principles, which act as germicides, enzyme inhibitors, odor absorbers or odor masks.

As germicides, which can be optionally added to the cosmetics according to the invention, basically all substances that are active against gram-positive bacteria are suitable, such as e.g. 4-hydroxybenzoic acid and its salts and esters, N-(4-chlorophenyl)-N′-(3,4-dichlorophenyl) urea, 2,4,4′-trichloro-2′-hydroxydiphenyl ether (Triclosan), 4-chloro-3,5-dimethylphenol, 2,2′-methylene-bis(6-bromo-4-chlorophenol), 3-methyl-4-(1-methylethyl) phenol, 2-benzyl-4-chlorophenol, 3-(4-chlorophenoxy)-1,2-propanediol, 3-iodo-2-propinylbutyl carbamate, chlorhexidine, 3,4,4′-trichlorocarbanilide (TTC), antibacterial fragrances, menthol, mint oil, phenoxyethanol, glycerin monolaurate (GML), diglycerin monocaprinate (DMC), salicylic acid-N-alkylamides such as, e.g. salicylic acid n-octylamide or salicylic acid n-decylamide.

Enzyme inhibitors can also be added to the cosmetics according to the invention. Examples of possible suitable enzyme inhibitors are esterase inhibitors. Trialkyl citrates are preferred, such as trimethyl citrate, tripropyl citrate, triisopropyl citrate, tributyl citrate and particularly triethyl citrate (Hydagen® CAT, Henkel KgaA, Düsseldorf/Germany). The substances inhibit the enzymatic activity and thereby reduce the odor formation. Additional substances that can be considered as esterase inhibitors are sterol sulfates or phosphates, such as e.g. lanosterin-, cholesterin-, campesterin-, stigmasterin- and sitosterin sulfate or phosphate, dicarboxylic acids and their esters, such as e.g. glutaric acid, monoethyl glutarate, diethyl glutarate, adipic acid, monoethyl adipate, diethyl adipate, malonic acid and diethyl malonate, hydroxycarboxylic acids and their esters such as e.g. citric acid, malic acid, tartaric acid or diethyl tartrate, as well as zinc glycinate.

Suitable odor absorbers are substances, which take up the odor forming compounds and firmly block them. They reduce the partial pressures of the individual components and thus also reduce their rate of propagation. It is important that the perfumes remain unaffected by this. Odor absorbers have no activity against bacteria. The comprise as the major component, for example, a complex zinc salt of ricinoleic acid or special, largely odor-neutral fragrances, which are known to the expert as fixing agents, such as e.g. extracts of labdanum or styrax or specific abietic acid derivatives. Fragrances or perfume oils act as masking agents and in addition to their function as masking agents, lend the deodorants their particular fragrance. Exemplary perfume oils include mixtures of natural and synthetic aromas. Natural aromas are extracts of flowers, stalks and leaves, fruits, fruit skins, roots, branches, herbs and grasses, needles and twigs as well as resins and balsams. In addition, animal materials such as e.g. civet and castoreum can be considered. Typical synthetic aroma compounds are products of the type of the esters, ethers, aldehydes, ketones, alcohols and hydrocarbons.

Antiperspirants reduce sweat formation by influencing the activity of the ecrinal sweat glands and thereby act against armpit moisture and body odor. Aqueous or anhydrous formulations of antiperspirants typically contain the following ingredients:
astringent principles,
oil components
nonionic emulsifiers,
co emulsifiers,
structurants,
auxiliaries such as e.g. thickeners or complexing agents and/or
non-aqueous solvents such as e.g. ethanol, propylene glycol and/or glycerin.

Salts of aluminum, zirconium or zinc are the main suitable astringent antiperspirant active principles. Such suitable antihydrotically active substances are e.g. aluminum chloride, hydrated aluminum chloride, hydrated aluminum dichloride, hydrated aluminum sesquichloride and their complexes e.g. with 1,2-propylene glycol, aluminum hydroxy allantoinate, aluminum chloride tartrate, aluminum-zirconium-trichlorohydrate, aluminum-zirconium tetrachlorohydrate, aluminum-zirconium pentachloro hydrate and their complexes e.g. with amino acids such as glycine.

The antiperspirants can also comprise standard oil-soluble and water-soluble auxiliaries in minor amounts. Such oil-soluble auxiliaries can be for example:
anti-inflammatory, skin protecting or fragrant essential oils,
synthetic skin-protecting active substances and/or
oil-soluble perfume oils.

Typical water-soluble additives are e.g. conservers, water-soluble aromas, pH adjustors, e.g. buffer mixtures, water-soluble thickeners, e.g. water-soluble natural or synthetic polymers such as e.g. xanthane gum, hydroxyethyl cellulose, polyvinyl pyrrolidone or high-molecular polyethylene oxides.

Climbazole, octopirox and zinc pyrethion can be used as anti-dandruff agents.

Usable film builders are for example, chitosan, microcrystalline chitosan, quaternized chitosan, polyvinyl pyrrolidone, vinyl pyrrolidone-vinyl acetate copolymers, polymers of the acrylic acid series, quaternized cellulose derivatives, collagen, hyaluronic acid or its salts and similar compounds.

As swelling agents for the aqueous phase, montmorillonite, mineral clays, Pemulen® as well as Carbopol types (Goodrich) can be used. Additional suitable polymers or swelling agents can be found in the review by R. Lochhead in Cosm. Toil. 108, 95 (1993).

The UV light protective factors are understood for example to be organic substances (protective light filters) that are liquid or solid at room temperature and which are able to absorb UV radiation and emit the resulting energy in the form of longer wavelength radiation, for example as heat. UVB filters can be oil-soluble or water-soluble. As oil-soluble substances, the following may be cited:
3-benzylidenecamphor or 3-benzylidenenorcamphor and its derivatives, for example 3-(4-methylbenzylidene) camphor, as described in the EP 0693471 B 1;
4-aminobenzoic acid derivatives, preferably 4-(dimethylamino)benzoic acid, 2-ethylhexyl ester, 4-(dimethylamino)benzoic acid, 2-octyl ester and 4-(dimethylamino)benzoic acid, amyl ester;
esters of cinnamic acid, preferably 4-methoxycinnamic acid, 2-ethylhexyl ester, 4-methoxycinnamic acid, propyl ester, 4-methoxycinnamic acid, isoamyl ester, 2-cyano-3,3-phenylcinnamic acid, 2-ethylhexyl ester (octocrylene);
esters of salicylic acid, preferably salicylic acid, 2-ethylhexyl ester, salicylic acid, 4-isopropylbenzyl ester, salicylic acid, homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzmalonic acid, di-2-ethylhexylester;
triazine derivatives, such as, for example 2,4,6-trianilino-(p-carbo-2′-ethyl-1′-hexyloxy)-1,3,5-triazine and octyl triazone, as described in EP 0818450 A1 or dioctyl butamidotriazone (Uvasorb® HEB);
propane-1,3-dione, such as for example 1-(4-tert.-butylphenyl)-3-(4′-methoxyphenyl)propane-1,3-dione;
ketotricyclo(5.2.1.0) decane derivatives, as described in EP 0694521 B1.

Water-soluble substances include: 2-phenylbenzimidazole-5-sulfonic acid and its alkali-, earth alkali-, ammonium-, alkylammonium-, alkanolammonium- and glucammonium salts;
sulfonic acid derivatives of benzophenones, preferably 2-hydroxy-4-methoxybenzophenone-5-sulfonic acid and its salts;
sulfonic acid derivatives of 3-benzylidenecamphor, as for example 4-(2-oxo-3-bornylidenemethyl)benzene sulfonic acid and 2-methyl-5-(2-oxo-3-bornylidene) sulfonic acid and its salts.

Typical UV-A filters particularly include derivatives of benzoylmethane, such as, for example 1-(4′-tert.-butylphenyl)-3-(4′-methoxyphenyl)propane-1,3-dione, 4-tert.-butyl-4′-methoxydibenzoylmethane (Parsol 1789), 1-phenyl-3-(4′-isopropylphenyl)-propane-1,3-dione as well as enamine compounds, as described in the DE 19712033 A1 (BASF). Naturally, the UV-A and UV-B filters can also be added as mixtures. Beside the cited soluble materials, also insoluble, light protective pigments, namely finely dispersed, preferably, nano metal oxides or salts can be considered for this task. Exemplary suitable metal oxides are particularly zinc oxide and titanium oxide and also oxides of iron, zirconium, silicon, manganese, aluminum and cerium as well as their mixtures. Silicates (talc), barium sulfate or zinc stearate cab be added as salts. The oxides and salts are already used in the form of pigments for skin care and skin protecting emulsions and decorative cosmetics. Here, the particles should have an average diameter of less than 100 nm, preferably between 5 and 50 nm and especially between 15 and 30 nm. They can be spherical, however elliptical or other shaped particles can be used. The pigments can also be surface treated, i.e. hydrophilized or hydrophobized. Typical examples are coated titanium dioxides, such as, for example Titandioxid Z 805 (Degussa) or Eusolex® T2000 (Merck); hydrophobic coating agents preferably include trialkoxy octylsilanes or Simethicones. Sun-protection agents preferably contain micropigments or nano-pigments. Micronized zinc oxide is preferably used. Further suitable UV light protection filters may be found in the review by P. Finkel in SoFW-Journal, Volume 122 (1996), p. 543.

As well as both above-cited groups of primary light protective materials, secondary light protective agents of the antioxidant type can also be used, which interrupt photochemical chain reactions that are propagated when the UV-radiation penetrates the skin. Typical examples are amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), carotinoides, carotines (e.g. α-carotine, β-carotine, lycopine) and their derivatives, chlorogenic acid and their derivatives, liponic acid and their derivatives (e.g. dihydroliponic acid), aurothioglucose, propylthiouracil and other thioles (e.g. thioredoxine, glutathione, cystein, cystine, cystamine and their glycosyl-, n-acetyl-, methyl-, ethyl-, propyl-, amyl-, butyl- and lauryl-, palmitoyl-, oleyl-, γ-linoleyl-, cholesteryl- and glyceryl esters) as well as their salts, dilauryl thiodipropionate, distearyl thiodipropionate, thiodipropionic acid and their derivatives (esters, ethers, peptides, lipids, nucleotides, nucleosides and salts) as well as sulfoximine compounds (e.g. buthioninesulfoximines, homocysteinsulfoximine, butionine sulfone, penta-, hexa-, heptathionine sulfoximine) in very minor compatible doses (e.g. pmol to μmol/kg), further (metal)-chelates (e.g. α-hydroxyfatty acids, palmitic acid, phytinic acid, lactoferrin), α-hydroxyacids (e.g. citric acid, lactic acid, malic acid), humic acid, gallic acid, gall extracts, bilirubin, biliverdin, EDTA, EGTA and their derivatives, unsaturated fatty acids and their derivatives (e.g. γ-linolenic acid, linolic acid, oleic acid), folic acid and their derivatives, ubiquinone and ubiquinol and their derivatives, vitamin C and derivatives (e.g. ascorbyl palmitate, Mg-ascorbyl phosphate, ascorbyl acetate), tocopheroles and derivatives (e.g. vitamin-E-acetate), vitamin A and derivatives (vitamin-A-palmitate) as well as coniferyl benzoate of benzoic resin, rutinic acid and their derivatives, α-glycosylrutine, ferula acid, furfurylideneglucitol, carnosine, butylhydroxytoluene, butylhydroxyanisol, nordihydroguajac resin acid, nordihydroguajaret acid, trihydroxybutyrophenone, uric acid and their derivatives, mannoses and their derivatives, superoxide-dismutase, zinc und its derivatives (e.g. ZnO, ZnSO₄) selenium and its derivatives (e.g. selenium-methionine), stilbenes and their derivatives (e.g. stilbene oxide, trans-stilbene oxide) and the suitable derivatives according to the invention (salts, esters, ethers, sugars, nucleotides, nucleosides, peptides and lipids) of these cited active substances.

To improve the flow properties, hydrotropes can also be added, such as, for example, ethanol, isopropyl alcohol, or polyols. Polyols, which are considered, possess preferably 2 to 15 carbon atoms and at least two hydroxyl groups. The polyols can comprise further functional groups, especially amino groups, or can be modified by nitrogen. Typical examples are
glycerin;
alkylene glycols, such as, for example ethylene glycol, diethylene glycol, propylene glycol, butylene glycol, hexylene glycol as well as polyethylene glycols with an average molecular weight of 100 to 1000 daltons;
technical oligoglycerin mixtures with a self condensation degree of 1.5 to 10 about like technical diglycerin mixtures with a diglycerin content of 40 to 50 wt. %;
methylol compounds, particularly like trimethylolethane, trimethylolpropane, trimethylolbutane, pentaerythritol and dipentaerythritol;
lower alkyl glucosides, particularly those with 1 bis 8 carbons in the alkyl radical, such as, for example methyl- and butyl glucoside;
sugar alcohols having 5 to 12 carbon atoms, such as, for example sorbitol or mannitol,
sugars with 5 to 12 carbon atoms, such as, for example glucose or saccharose;
aminosugars, such as, for example glucamine;
dialcoholamines, like diethanolamine or 2-amino-1,3-propanediol.

Suitable preservatives are, for example phenoxyethanol, formaldehyde solution, parabene, pentanediol or sorbic acid as well as the further classes of materials described in Appendix 6, part A and B of the Cosmetic Regulation. Insect repellants include N,N-diethyl-m-toluamide, 1,2-pentanediol or ethyl butylacetylaminopropionate; suitable self tanning agents include dihydroxyacetone.

As perfume oils, the known mixtures of natural and synthetic aromas can be cited Natural aromas are extracts of flowers (lilies, lavender, roses, jasmine, neroli, ylang ylang), stalks and leaves (geranium, patchouli, petit grain), fruits (aniseed, coriander, caraway, juniper), fruit skins (bergamot, lemons, oranges), roots (mace, angelica, celery, cardamom, costic, iris, calmus), wood (pine, sandal, guava, cedar, rose wood), herbs and grasses (tarragon, lemon grass, sage, thyme), needles and twigs (spruce, fir, scotch pine, larch), resins and balsam (galbanum, elemi, benzoin, myrrh, olibanum, opoponax). In addition, animal raw materials can be considered, such as civet and castoreum. Typical synthetic aroma compounds are products of the ester, ether, aldehyde, ketone, alcohol and hydrocarbon types.

As colorants, those substances suitable and approved for cosmetic purposes can be used, as summarized, for example in the publication “Kosmetische Färbemittel” of the Colorant Commission of the Deutsche Forschungsgemeinschaft, Verlag Chemie, Weinheim, 1984, p. 81-106. These colorants are typically used in concentrations of 0.001 to 0.1 wt. %, based on the total mixture.

The total content of auxiliaries and additives can be 1 to 50, preferably 5 to 40 wt. %, based on the agent. The manufacture of the agent can be made using customary cold or hot processes; preferably according to the phase inversion temperature method.

A further subject of the invention is an oxidative colorant for dyeing keratin fibers, comprising choline oxidases used according to the invention and particularly choline oxidases according to the invention. Keratin fibers are understood to mean wool, feathers, skins and particularly human hair.

For the manufacture of the oxidizing agents according to the invention, the oxidative colorant precursors, together with the choline oxidases are mixed together in a suitable aqueous carrier under the exclusion of oxygen from the air. Such carriers are e.g. thickened aqueous solutions, creams (emulsions), gels or surfactant-containing foam preparations, e.g. shampoos or foam aerosols or other preparations that are suitable for application on hair.

Basically, anhydrous powders are also suitable as carriers; in this case, the oxidative colorants are dispersed or dissolved in water immediately before use. Preferred components of carriers that are used are
wetting agents and emulsifiers
thickeners
reducing agents (antioxidants)
hair care additives
fragrances and
solvents such as e.g. water, glycols or lower alcohols.

Suitable wetting agents and emulsifiers are e.g. anionic, zwitterionic, ampholytic and nonionic surfactants. Cationic surfactants can also be used to obtain specific effects.

Suitable thickeners are the water-soluble high-molecular polysaccharide derivatives or polypeptides, e.g. cellulose ethers or starch ethers, gelatin, plant gums, biopolymers (xanthane gum) or water-soluble synthetic polymers such as e.g. polyvinyl pyrrolidone, polyvinyl alcohol, polyethylene oxides, polyacrylamides, polyurethanes, polyacrylates and others.

In addition, surfactant-containing preparations can also be thickened by solubilization or emulsification of polar lipids. Such lipids are e.g. fatty alcohols with 12-18 carbon atoms, (free) fatty acids with 12-18 carbon atoms, partial glycerides of fatty acids, sorbitol esters of fatty acids, fatty acid alkanolamides, lower oxyethylated fatty acids or fatty alcohols, lecithin, sterin. Finally, carriers in gel form can also be produced on the basis of aqueous soap gels e.g. ammonium oleate.

Reducing agents (antioxidants), which are added to the carrier, in order to prevent a premature oxidative development of the colorant before its use on the hair, are e.g. sodium sulfate or sodium ascorbate.

Hair care ingredients can include, for example, fats, oils or waxes in emulsified form, structuring additives, such as, for example, glucose or pyridoxine, brightening components, for example, water-soluble proteins, protein degradation products, amino acids, water-soluble cationic polymers, silicones, vitamins, panthenol or plant extracts.

Finally, fragrances and solvents, for example, glycols such as 1,2-propylene glycol, glycerol, gycol ethers such as butyl glycol, ethyl diglycol or lower monohydroxy alcohols such as ethanol or isopropanol, can also be ingredients.

In addition, other additives can also be present in order to improve the stability and usage properties of the oxidizing dyes, for example, complexing agents such as EDTA, NTA or organophosphonates, swelling and penetrating agents, such as, for example urea, guanidine, bicarbonates, buffer salts such as, for example, ammonium chloride, ammonium nitrate, ammonium sulfate or alkyl ammonium salts and propellants if required.

A further subject of the invention is as an agent for the care of the mouth, teeth or dentures, especially denture cleaners, containing choline oxidase usable according to the invention, especially choline oxidase according to the invention for bleaching or for disinfection.

In the case of partial prostheses or dentures, presentation as denture cleaning tablets or as mouthwash or rinse or toothpaste is also suitable.

The mouth, tooth or denture care agents according to the invention can be available for example as a mouthwash, gel, liquid tooth cleaner, firm toothpaste, denture cleaner or denture adhesive cream.

For this, the choline oxidase usable according to the invention and especially choline oxidase according to the invention, need to be incorporated within a suitable carrier.

For example, preparations in powder form or water-alcohol solutions can serve as carriers, which can contain 0 to 15 weight % ethanol, 1 to 1.5 weight % flavor oils and 0.01 to 0.5 weight % sweeteners for mouthwash or 15 to 60 weight % ethanol, 0.05 to 5 weight % flavor oils, 0.1 to 3 weight % sweeteners as well as other additives if required for mouthwash concentrate which is diluted with water before use. The concentration of the components must be selected at a high enough level, such that following dilution the concentration during use does not fall below the lower limit of the range mentioned.

Gels as well as more or less flowable pastes, which are squeezed out of flexible plastic containers or tubes and applied to the teeth with the aid of a toothbrush, can also serve as carriers. Such products contain higher quantities of wetting and binding agents or stabilizers and polishing agents. Furthermore, perfume oils, sweeteners and water also contained in these preparations.

Wetting agents can include, for example, glycerol, sorbitol, xylite, propylene glycols, polyethylene glycols or mixtures of these polyols, especially polyethylene glycols with a molecular weight from 200 to 800 (from 400-2000). The preferred wetting agent is sorbitol in a quantity of 25-40 weight %.

Condensed phosphates in the form of their alkali salts, preferably in the form of the sodium or potassium salt, can be included as anti-tartar agents and as inhibitors of demineralization. Aqueous solutions of these phosphates react as alkalis due to hydrolytic effects. On addition of acid, the pH of the mouth, tooth and/or denture care agents according to the invention is kept within the preferred range of 7.5-9.

A mixture of various condensed phosphates or also hydrated salts of condensed phosphates can be used. However, the specified quantities of 2-12 weight % refer to the anhydrous salts. The preferred condensed phosphate is a sodium or potassium tripolyphosphate in a quantity of 5-10% by weight of the composition.

A favored active ingredient is a caries-inhibiting fluorine compound, preferably from the fluoride or monofluorophosphate group in a quantity of 0.1-0.5 weight % fluorine. Suitable fluorine compounds include, for example, sodium monofluorophosphate (Na₂PO₃F), potassium monofluorophosphate, sodium or potassium fluoride, stannous fluoride or a fluoride of an organic amino compound.

Binding agents and stabilizers include, for example, natural and synthetic water-soluble polymers such as carrageenan, traganth, guar, starch and their non-ionic derivatives such as hydroxypropyl guar, hydroxyethyl starch, cellulose ethers such as hydroxyethyl cellulose or methylhydroxypropyl cellulose. Agar-agar, xanthan gum, pectins, water-soluble carboxyvinyl polymers (e.g. Carbopol® types), polyvinyl alcohol, polyvinyl pyrrolidone, high-molecular-weight polyethylene glycols (molecular weight 10³ to 10⁶ D) can also be used. Other materials suitable for controling viscosity include layered silicates, for example montmorillonite, colloidal swelling silicas, e.g. aerogel silicas or pyrogenic silicas.

Polishing components can include all polishing agents that are known for this purpose, but preferably precipitated and gel silicas, aluminum hydroxide, aluminum silicate, aluminum oxide, aluminum oxide trihydrate, insoluble sodium metaphosphate, calcium pyrophosphate, dicalcium phosphate, chalk, hydroxyapatite, hydrotalcite, talcum, magnesium aluminum silicate (Veegum®), calcium sulfate, magnesium carbonate, magnesium oxide, sodium aluminum silicates, for example zeolite A or organic polymers, for example polymethacrylate. The polishing agents are preferably used in smaller quantities, for example 1-10 weight %.

The tooth and/or mouth care products according to the invention can have their organoleptic qualities improved by the addition of flavor oils and sweeteners. Flavor oils can include all natural and synthetic flavorings used in products for the care of mouth, teeth or dentures. Natural flavors can be used in the form of etheric oils isolated from the drugs or in the form of individual components isolated from the latter. Preferably, at least one flavor oil from the group of peppermint oil, curled mint oil, anise oil, caraway oil, eucalyptus oil, fennel oil, cinnamon oil, geranium oil, sage oil, thyme oil, marjoram oil, basil oil, citrus oil, wintergreen oil, or one or more synthetically produced components isolated from these oils should be included. The most important components of the abovementioned oils are, for example, menthol, carol, anethole, cineol, eugenol, cinnamaldehyde, geraniol, citronellol, linalool, salvene, thymol, terpinene, terpinol, methyl chavicol and methyl salicylate. Other suitable flavorings include, for example, menthyl acetate, vanillin, ionone, linalyl acetate, rhodinol and piperitone. Natural sugars such as sucrose, maltose, lactose and fructose or synthetic sweeteners such as sodium saccharin, sodium cyclamate or aspartame can be used for sweetening.

Surfactants that can be used include alkyl- and/or alkenyl-(oligo)-glycosides in particular. Their manufacture and use as surfactants is well known for example from U.S. Pat. No. 3,839,318, U.S. Pat. No. 3,707,535, U.S. Pat. No. 3,547,828 DE-A-19 43 689, DE-A-20 36472 and DE-A-30 01 064 as well as from EP-A-77 167. With respect to glycoside residues, both monoglycosides (x=1), in which a pentose or hexose residue is glycosidically bound to a primary alcohol of 4-16 C atoms, and oligomeric glycosides with a degree of oligomerization of up to 10 are suitable. In this case the degree of oligomerization is a statistical average, which in such technical products is usually based on a homologous distribution.

Suitable alkyl and/or alkenyl-(oligo)-glycosides are preferably of the formula RO(C₆H₁₀O)ₙ—H, in which R is an alkyl-and/or alkenyl group with 8 to 14 C atoms and x has an average value of 1 to 4. Alkyl-oligo-glucosides based on hydrogenated C_{12/14} coconut alcohol with a DP of 1 to 3 are especially suitable. Alkyl- and/or alkenyl-glycoside surfactants can be used very economically, amounts up to 1 weight % being sufficient.

In addition to the abovementioned alkyl glucoside surfactants, other non-ionic, amphoteric and cationic surfactants can be included, such as: fatty alcohol polyglycol ether sulfates, monoglyceride sulfates, monoglyceride ether sulfates, mono- and/or dialkyl sulfosuccinates, fatty acid isothionates, fatty acid sarcosinates, fatty acid taurides, fatty acid glutamates, ether carboxylic acids, fatty acid glucamides, alkyl amido betaine and/or protein/fatty acid condensates, the latter preferably being based on wheat proteins. Especially when dissolving the mainly water-insoluble flavor oils, one may need to use a non-ionic solvent from the surfactant group. Oxyethylated fatty acid glycerides, oxyethylated fatty acid sorbitol partial esters or fatty acid partial esters of glycerol or sorbitol oxethylates are especially suitable for this purpose. Solvents from the oxethylated fatty acid glyceride group include, above all, all the addition products of 20 to 60 moles ethylene oxide with mono- and diglycerides of linear fatty acids having 12 to 18 C atoms or with triglycerides of hydroxy fatty acids such as oxystearic acid or ricinoleic acid. Other suitable solvents include oxyethylated fatty acid sorbitol partial esters; preferably adsorption products from 20 to 60 mol ethylene oxide with sorbitol monoesters and sorbitol diesters of fatty acids having 12 to 18 C atoms. Fatty acid partial esters of glycerol and sorbitol oxyethylates are also suitable solvents, preferably mono- and diesters of C₁₂-C₁₈ fatty acids and addition products of 20 to 60 moles ethylene with 1 mole glycerol or 1 mole sorbitol.

Mouth, tooth and/or denture care products according to the invention preferably comprise as solubilizing agents for the optionally included flavor oils, addition products of 20 to 60 moles ethylene oxide with hydrogenated or non-hydrogenated castor oil (namely with triglyceride of oxystearic or ricinoleic acid), with glycerol mono- and/or distearate or with sorbitol mono- and/or distearate.

Examples of other customary additives to mouth, tooth and denture care products include,
pigments, e.g. titanium dioxide, and/or colorings
pH adjusters and buffers, e.g. sodium bicarbonate, sodium citrate, sodium benzoate, citric acid, phosphoric acid or acid salts, e.g. NaH₂PO₄
wound-healing and anti-inflammatory substances, e.g. allantoin, urea, panthenol, azulene or camomile extract
other anti-tartar substances e.g. organophosphates such as hydroxyethane diphosphonate or azacycloheptane diphosphonate
preservatives, e.g. sorbic acid salts, p-hydroxybenzoic acid ester.
plaque inhibitors such as hexachlorophene, chlorhexidine, hexetidine, triclosan, bromchlorophene, phenylsalicylic acid ester.

In a specific embodiment, the composition is a mouthwash, a rinse, a denture cleaner or dental adhesive.

For a preferred denture cleaner according to the invention, especially for denture-cleaning tablets and powder, in addition to the already listed ingredients for mouth, tooth and/or denture care products, per-compounds such as peroxyborate, peroxymonosulfate or percarbonate are also suitable. They have the advantage that in addition to a bleaching effect they simultaneously act to deodorize and/or disinfect. Such per-compounds are used in denture cleaners at between 0.01 and 10 weight %, and especially at between 0.5 and 5 weight %.

Enzymes, e.g. proteases and carbohydrase, are also suitable ingredients for the degradation of proteins and carbohydrates. The pH value can be in the range pH 4 to 12, and especially in the range pH 5 to pH 11.

Still other additives are required for denture cleaning tablets, for example agents that cause effervescence, e.g. CO₂-liberating substances such as sodium bicarbonate, fillers such as sodium sulfate or dextrose, lubricants, e.g. magnesium stearate, flow regulators such as colloidal silicon dioxide and granulating agents such as the already mentioned high-molecular-weight polyethylene glycols or polyvinyl pyrrolidone.

Denture fixative agents can be offered as powders, creams, films or liquids and they assist denture adhesion.

Materials from natural and synthetic sources are suitable as the active agents. In addition to alginates, natural materials include plant gums such as gum arabic, traganth gum and karaya gum as well as natural rubber. Particular use is made of alginates and synthetic materials such as sodium carboxymethylcellulose, high-molecular-weight ethylene oxide copolymers, salts of vinyl ether-maleic acid copolymer, and polyacrylamide.

Hydrophobic bases are particularly suitable as additives for paste and liquid products, especially hydrocarbons such as white vaseline (DAB—German Pharmacopoeia) or mineral oil.

A further subject of the invention is a signaling reagent for the production of a light emission in a chemiluminescence assay, comprising choline oxidase according to the invention, a choline oxidase substrate and a chemiluminescent reagent. Choline is the preferred choline oxidase substrate. Luminol is the preferred chemiluminescent reagent.

Chemiluminescence tests are of great significance in the fields of medicine and the life sciences. Immunoassays and DNA probe analyses are especially important. The advantage of choline oxidases according to the invention, which produce oxygen containing free radicals (e.g. superoxide anions, hydroxyl radicals) and peroxides (hydrogen peroxide), is that they supply long-lived products, which can be detected by chemiluminescence, in the reaction with chemiluminescent reagents such as lucigenin, luminol and their derivatives. These choline oxidase systems are especially useful as tracers for the detection of analytes by means of immunoassays, immunoblotting or nucleotide probe analyses as a means of supplying longer-lived, light-emitting entities following reaction with a chemiluminescent reagent.

A further subject of the invention is the use of a choline oxidase used according to the invention, especially a choline oxidase according to the invention, for the production of betaine, for the production of foodstuffs and or food components and also for the production of animal feeds and/or animal feed components.

Betaine is of importance as a conditioning component in shampoos and hair care products as well as in detergents. It is also used as a food supplement in animal feeds, since it has a protective effect on the liver.

The following examples describe the invention without however restricting it:

### EXAMPLE 1

### Microbiological screening

Enrichment cultures were obtained from soil samples. The selection criterion was growth on agar plates containing choline as the sole source of C. The Arthrobacter KC2 strain was found among the candidates and was deposited at the DSMZ (German Collection of Microorganisms and Cell Cultures) (DSMZ-ID 96-878)

### EXAMPLE 2

### Isolation of Choline Oxidase from the Arthrobacter nicotianiae Strain (KC2)

Culturing took place in a yeast medium with the following composition:

1.0 g/l K₂HPO₄, 0.5 g/l NH₄CI, 0.2 μl MgSO₄×7H₂O, 0.01 g/l CaCl₂×2H₂O, 0.8 ml trace salt solution, 0.5 g/l yeast extract, 5.0 μl choline chloride, 0.65 M NaCl adjusted to a pH of pH=8.0. The incubation took place for about 27 hours at 30° C. and 120 rpm. Subsequently, the cells were harvested by centrifugation and homogenized in a glass bead beater (30% cell suspension). After a second centrifugation, the raw extract containing the choline oxidase was obtained as the supernatant.

Purification:

First the raw extract was buffered in Buffer A (20 mM imidazole, pH=7.0) over a PD-10 desalting column (Amersham Pharmacia Biotech, Cat no. 17-0851-01) according to the protocol recommended by the manufacturer. Then the choline oxidase-containing solution was fractionated on an ion exchange column (Q-Sepharose, Amersham Pharmacia Biotech, Cat no. 17-1014-01) with a column volume of 16.5 ml at a flow rate of 2 ml/min and under a pressure of 0.3 MPa and then rinsed with 2 column volumes of Buffer A under the same conditions. The elution was carried out with 5 column volumes of Buffer B (20 mM imidazole+1M NaCl, pH=7.0) across a linear NaCl gradient (0-100%) under the conditions described above. The choline oxidase-containing fraction was concentrated and buffered in Buffer C (20 mM Tris-HCl, pH=7.6) on a PD-10 column according to the protocol suggested by the manufacturer. Then the choline oxidase-containing solution was fractionated on an ion exchange column (Resource Q, Amersham Pharmacia Biotech, Cat nor 17-1179-01) with a column volume of 6 ml at a flow rate of 6 ml/min and under a pressure of 1.2 MPa and was rinsed in 5 column volumes of Buffer C under the same conditions. Elution was carried out with 20 column volumes of Buffer D (20 mM Tris-HCl+1 M NaCl, pH=7.6) across a linear NaCl gradient (0-100%) under the conditions described above.

### EXAMPLE 3

### Gene Isolation

The DNA probe (Seq. 6) was generated using PCR, the primers (Seq. 7 and 8) being constructed on the basis of the known sequence of choline oxidase from Arthrobacter globiformis, the primers are located within the gene. The probe comprises 900 bp and was cut out with restriction enzymes (Fspl and Styl) from the DNA fragment obtained.

### EXAMPLE 4

### Cloning Choline Oxidase KC2

Sequences of the primers used:

Forward primer: (Seq. 8)
KC2pETNcoF:
5′ - CATGCCATGGCAAAGGGCGAAAAATTGAACATTG - 3′
Reverse primer: (Seq. 9)
KC2pETHindR:
5′ - CCCAAGCTTCTAGGCTTCGCTAACCAGTTCG - 3′

Expression system: vector:

Vector: pET 26 b+from Novagen; Cat. no. 69862-3

Host strain: BL 21 Gold (DE 3) from Stratagene; Cat. no.:230132

### EXAMPLE 5

### Creation of a Hybrid Enzyme.

The hybrid gene consists of the choline oxidase gene of KC2 and the choline oxidase gene of Arthrobacter aurescens. Isolation of the Arthrobacter aurescens gene was also carried out using a DNA probe.

Composition of the gene:
bases 1-269: from Arthrobacter aurescens
bases 267-1171: from KC 2
bases 1172-1629: from Arthrobacter aurescens

### EXAMPLE 6

### Biochemical Characterization

Specific activity: Activity test for choline oxidases (4-aminoanfipyrin/chromotropic acid)

1. Solutions

The following solutions were required for the determination of choline oxidase activity:

| Peroxide | K₂PO₄ | 75 | mM | 0.68 | g/50 ml |
|---|---|---|---|---|---|
| reagent: | | | | | |
| | NaH₂PO₄ | 125 | mM | 0.89 | g/50 ml |
| | chromotropic acid | 12.5 | mM | 0.2503 | g/50 ml |
| | 4-Aminoantipyrine | 0.6 | mM | 6 | mg/50 ml |
| Phosphate | K₂PO₄ | 75 | mM | 13.6 | g/l |
| buffer: | | | | | |
| pH = 6.5 | NaH₂PO₄ | 125 | mM | 17.8 | g/l |
| Peroxidase: | 50 U/mL horseradish peroxidase in phosphate buffer | | | | |
| Davis Buffer: | citric acid × 1H₂O | 0.1 | M | 21.01 | g/l |
| stock | NaB₄O₇ × 1H₂O | 0.1 | M | 19.07 | g/l |
| solution | KCl | 0.1 | M | 7.46 | g/l |
| | KH₂PO₄ | | | 13.61 | g/l |
| | tris-(hydroxy- | 0.1 | M | 12.11 | g/l |
| | aminomethane | | | | |

| | |
|---|---|
| | (diluted 1:4 with distilled water and adjusted to pH 9.5 |
| | with 0.4 M NaOH) |
| 20 mM NaN₃: | 65 mg/50 ml in Davis buffer pH = 9.5 |

250 mM choline chloride in distilled H₂O

2. Method:

To 150 μl of choline oxidase solution were added 30 μl of 20 mM NaN₃ solution, 45 μl of Davis buffer pH=9.5 and 150 μl of 250 mM choline chloride and after mixing, the preparation was incubated for 30 min at 800 rpm and 37° C. Then 525 μl of peroxide reagent and 75 μl of peroxide solution were added and the mixture incubated for 5 min at room temperature. Finally the absorption of the solution was determined at a wavelength λ=600 nm.

Activity was determined by means of a comparison with a calibration curve that has been established with an appropriate dilution series of hydrogen peroxide in 20 mM phosphate buffer (pH 6.5) under the conditions described above.

The quantity of protein per sample volume was determined using the BCA Protein Assay Kit (Pierce Biotechnology, Cat. no. 23227) according to the protocol suggested by the manufacturer and expressed in mg/μl. The specific activity is defined as the activity per mg protein and is expressed in U/mg or mU/mg=10⁻³ U/mg. The specific activity of KC 2 here refers to the sample volume in the activity test and averages 41 mU/mg for the raw extract (mechanical dissociation) and 123 mU/mg after purification.

pH Stability:

Choline oxidase is stable in a pH range of 6-12, especially at a pH of 8-10.

Method:

The purified oxidase was diluted 1:30 with Davis buffer at the corresponding pH and incubated for one hour at 30° C. and 800 rpm. Finally, an activity test was carried out on 150 μl of solution as described above.

Temperature Profile:

Choline oxidase is active in the temperature range from 10° C. to 70° C. and especially in the temperature range from 25° C. to 40° C. It is stable in the temperature range from 10° C. to 50° C. and especially in the temperature range from 10° C. to 40° C.

Method:

In order to determine activity at a defined temperature, the purified choline oxidase was diluted in Davis buffer pH 9.5 with sodium azide (corresponding to a final concentration of 3 mM). Determination of activity took place with 150 μl of solution in the same way as in the protocol described above, with the difference that incubation took place at the corresponding temperature instead of at 37° C.

For determination of temperature stability, the purified choline oxidase was diluted in Davis buffer pH 9.5 with sodium azide (corresponding to a final concentration of 3 mM), which had been preincubated at the corresponding temperature for one hour. Finally, an activity test was carried out on 150 μl of solution as described in the protocol above.

### EXAMPLE 7

### Production of Mutants with Improved Specific Activity for Choline

The KC2 was expressed in E. coli, to which a construct with Seq. 17 was produced. It differed on account of mutations C2T, A4G and G5C at the start of the gene and the silent mutation C1186A. Thus there was a difference in Seq. 1 and Seq. 10 in the amino acids TIM and R2A.

The mutants were variants of the choline oxidase KC2 in E. coli (Seq. 17) and were expressed in the same construct.

Mutation C1186A is a silent mutation, which is not found in Arthrobacter. It does however appear in E. coli expressing choline oxidase genes (KC2 in E. coli, KC2 M85V, KC2 A27V, KC2 M85V2 d. h. Seq. 17, 18, 19, 20).

The mutation C612T in the mutant KC2 M85V2 (Seq. 20) is also a silent mutation that does however result in improved expression. In the literature, such a phenomenon is termed an expression mutation. In the present case, the improved expression of KC2 M85V2 is recognized due to the higher specific activity of the raw extract.

The KC2s gene (Seq. 21) was obtained from Seq. 10 by cleaving away the first 18 bases of the KC2 gene (Seq. 17). Seq. 22 was produced for cloning and differed from Seq. 21 in base 1 (T1A). The resulting gene product is presented in Seq. 15.

In the same way, a “KC2s variant” can be produced from each of the mutations mentioned.

Summary of the mutants and their characteristics:

| | | Specific | | |
|---|---|---|---|---|
| | Specific activiy | activity, | Mutation, DNA | Mutation, |
| Choline oxidase | in raw extract | purified | level | protein level |
| KC2 from Arthrobacter | NA | NA | — | — |
| (Seq. 1 and Seq. 4) | | | | |
| KC2, heterologous from | 80.6 mU/mg | 123 mU/mg | C1186A | — |
| E. coli, (Seq. 10 and | | | | |
| Seq 17) | | | | |
| KC2 M85V | 112.6 mU/mg | 870 mU/mg | A253G, | M85V |
| (Seq. 11 and Seq. 18) | | | C1186A, | |
| | | | C1188A | |
| KC2 A27V | 132.6 mU/mg | 670 mU/mg | C80T, C1186A, | A27V |
| (Seq. 12 and 19) | | | C1254T, | |
| | | | C1369T | |
| KC2 M85V2 | 213.9 mU/mg | 920 mU/mg | A253G, C612T, | M85V |
| (Seq. 13 and 20) | | | C1186A, | |
| | | | C1188A | |
| KC2s | 70 mU/mg | 508 mU/mg | C1186A | |
| (Seq. 15 and Seq. 22) | | | | |

### EXAMPLE 8

### Production of KC2s (KC2s)

Sequences of the primers used

(Seq. 23)

KC2spETNF:
5′ TTCCATATGAACATTGAAAAGAAGGACTTCGACTACATTG 3′

(Seq. 9)

KC2pETHR

Amplification of the genomic DNA of Arthrobacter nicotianae or of the choline oxidase KC2 gene was carried out using an suitable PCR method.

Cloning took place in the NdeI-HindIII site of the pET 26 b+vector from Novagen (Cat. no. 69862-3).

Transformation was carried out by means of standard methods in E. coli BL21 Gold (DE3) (Stratagene, Cat. no. 230132)

### EXAMPLE 9

### Washing Study

Washing matrix: basic formulation as presented in the following table:

| | Basic formulation |
|---|---|
| Chem. Name | (% pure substance) |
| Xanthan gum | 0.3-0.5 |
| Antifoaming agent | 0.2-0.4 |
| Glycerol | 6-7 |
| Ethanol | 0.3-0.5 |
| FAEOS | 4-7 |
| Non-ionic surfactant (FAEO, APG, etc.) | 24-28 |
| Boric acid | 1 |
| Sodium citrate × 2H₂O | 1-2 |
| Caustic soda | 2-4 |
| Coconut fatty acid | 14-16 |
| HEDP | 0.5 |
| PVP | 0-0.4 |
| Optical brighteners | 0-0.05 |
| Colorings | 0-0.001 |
| Fragrance | 0-2 |
| H₂O, demin. | Rest |

Dosage: 4.4 g/l

Staining: tea on cotton, e.g. stain 020 J Co from wfk Testgewebe GmbH (Brüggen-Bracht, Germany), E-167 from EMPA Testmaterialien AG (St. Gallen, Switzerland) or HTB from Henkel KgaA (Düsseldorf, Germany).
Time: 30 min
pH during the procedure: 10 (adjusted with sodium hydroxide)
Evaluation: determination of the L-value

Method:

Test fabric pieces (diameter 10 mm) were incubated in a 24-well microtiter plate in 1 ml of detergent solution for 30 min at 37° C. with agitation at 100 rpm. The detergent contained 265 mU choline oxidase KC2 or an equivalent protein quantity of mutants or KC2s from the corresponding preparations. The substance concentrations were between 100 mM and 200 mM choline chloride. During the incubation, air was introduced into each sample via a tube with an inside diameter of 0.5 to 1 mm. Each sample was subjected to two-fold determination and compared with a two-fold determination in a control without choline oxidase.

After washing, the degree of whiteness of the washed textiles was measured in comparison with a whiteness standard (d/8, Ø 8 mm, SCI/SCE), which had been set at 100%. The measurement was made using a colorimeter (Minolta Cm508d) with a light setting of 10°/D65. The results obtained are presented in the following table in terms of percent remission, i.e. as a percentage in comparison with the white standard together with the respective starting values. The bleaching effect is given as ΔL, the difference in remission between the basic washing formulation with choline oxidase and a control without choline oxidase subjected to the same test conditions.

The effect of bleach activators

| Basic washing | TAED conc. | Choline conc. | Bleaching | Standard |
|---|---|---|---|---|
| formulation with: | in weight % | mmol/l | effect ΔL | deviation |
| KC2 | 0 | 200 | 3.40 | 0.28 |
| KC2 | 0.03 | 200 | 4.50 | 0.07 |

It can be seen that the presence of choline oxidase KC2 according to the invention in the basic formulation without TAED has a bleaching effect on the tested stain. This effect was strengthened by the addition of TAED.

Washing effect of mutants compared to KC2 and KC2s

| Basic washing | TAED conc. | Choline conc. | Bleaching | Standard |
|---|---|---|---|---|
| formulation with: | in weight % | mmol/l | effect ΔL | deviation |
| KC2 | 0.03 | 100 | 3.34 | 0.23 |
| KC2 M85V | 0.03 | 100 | 4.26 | 0.55 |
| (M85V) | | | | |
| KC2 A27V | 0.03 | 100 | 4.34 | 0.59 |
| (A27V) | | | | |
| KC2 M85V2 | 0.03 | 100 | 4.43 | 0.46 |
| (M85V) | | | | |
| KC2s | 0.03 | 100 | 3.57 | 0.32 |

The choline oxidase KC2 mutants according to the invention (KC2 M85V, KC2 A27V and KC2 M85V2) as well as KC2s showed a more pronounced bleaching effect than KC2 against tea stains in a basic formulation with TAED.

### EXAMPLE 10

### Comparison of the Bleaching Effect of Choline Oxidase with a Conventional Bleaching System

Washing matrix: as in Example 9
Dosage: 4.4 g/l
Staining: tea on cotton, e.g. stain 020 J Co from wfk Testgewebe GmbH (Brüggen-Bracht, Germany), E-167 from EMPA Testmaterialien AG (St. Gallen, Switzerland) or HTB from Henkel KgaA (Düsseldorf, Germany).
Time: 30 min
pH during the procedure: 10 (adjusted with sodium hydroxide)
Evaluation: determination of the L-value
Method:

Test fabric pieces (diameter 10 mm) were incubated in a 24-well microtiter plate in 1 ml of detergent solution for 30 min at 37° C. with agitation at 100 rpm. The detergent contained 265 mU choline oxidase KC2 and 200 mM choline chloride. During the incubation, air was introduced into each sample via a tube with an inside diameter of 0.5 to 1 mm. In order to determine the bleaching effect of chemical bleaches, 97 mg percarbonate was added to the detergent shortly before the test. This prevented the premature decomposition of the bleach. Each sample was subjected to two-fold determination and compared with a two-fold determination in a control without choline oxidase or percarbonate.

After washing, the degree of whiteness of the washed textiles was measured in comparison with a whiteness standard (d/8, Ø 8 mm, SCI/SCE), which had been set at 100%. The measurement was made using a colorimeter (Minolta Cm508d) with a light setting of 10°/D65. The results obtained are presented in the following table in terms of percent remission, i.e. as a percentage in comparison with the white standard together with the respective starting values. The bleaching effect is given as ΔL, the difference in remission between the basic washing formulation with choline oxidase and a control without choline oxidase subjected to the same test conditions.

A Comparison of Enzymatic and Chemical Bleaches

| Basic washing | TAED conc. | Choline conc. | Bleaching | Standard |
|---|---|---|---|---|
| formulation with: | in weight % | mmol/l | effect ΔL | deviation |
| Percarbonate. | 0.03 | 0 | 3.03 | 0.30 |
| KC2 | 0.03 | 200 | 4.50 | 0.28 |

It can be seen that the presence of choline oxidase KC2 according to the invention in the basic formulation with TAED has a stronger bleaching effect on the tested stains than a chemical bleach used in the standard manner.

### EXAMPLE 11

### Comparison of the Bleaching Effect of Choline Oxidase Against Various Bleachable Stains.

Washing matrix: As in Examples 9 and 10
Dosage: 4.4 g/l
Staining:
Tea on cotton, e.g. Stain 020 J Co from wfk Testgewebe GmbH (Brüggen-Bracht, Germany), E-167 from EMPA Testmaterialien AG (St. Gallen, Switzerland) or HTB from Henkel KGaA (Düsseldorf, Germany).
Blackberry on cotton, e.g. Stain 041 BB Co from wfk Testgewebe GmbH (Brüggen-Bracht, Germany), or HBBB from Henkel KGaA (Düsseldorf, Germany).
Red wine on cotton, e.g. E-1 14 from EMPA Testmaterialien AG (St. Gallen, Switzerland) or HRB from Henkel KGaA (Düsseldorf, Germany).
Time: 30 min
pH during the procedure: 10 (adjusted with sodium hydroxide)
Evaluation: determination of the L-value

Method:

Test fabric pieces (diameter 10 mm) were incubated in a 24-well microtiter plate in 1 ml of detergent solution for 30 min at 37° C. with agitation at 100 rpm. The detergent contained 265 mU choline oxidase KC2 and 200 mM choline chloride. During the incubation, air was introduced into each sample via a tube with an inside diameter of 0.5 to 1 mm. Each sample was subjected to two-fold determination for each stain and compared with a two-fold determination in a control without choline oxidase

After washing, the degree of whiteness of the washed textiles was measured in comparison with a whiteness standard (d/8, Ø 8 mm, SCI/SCE), which had been set at 100%. The measurement was made using a colorimeter (Minolta Cm508d) with a light setting of 10°/D65. The results obtained are presented in the following table in terms of percent remission, i.e. as a percentage in comparison with the white standard together with the respective starting values. The bleaching effect for each stain is given as ΔL, the difference in remission between the basic washing formulation with choline oxidase and a control without choline oxidase subjected to the same test conditions.

Comparison of enzymatic bleaches and various stains

| Basic washing | TAED conc. in | | Bleaching | Standard |
|---|---|---|---|---|
| formulation with: | weight % | Stain | effect ΔL | deviation |
| KC2 | 0.03 | Tea | 4.50 | 0.28 |
| KC2 | 0.03 | Blackberry | 1.40 | 0.35 |
| KC2 | 0.03 | Red wine | 2.00 | 0.14 |

It can be seen that under the conditions described, choline oxidase KC2 according to the invention has a bleaching effect against the relative stains.

1. A bodycare product; shampoo; hair care product; oral care product; tooth or denture care product; cosmetic; detergent; cleaning agent; rinsing agent; hand washing detergent; hand dishwashing detergent; machine dishwashing detergent; disinfectant; or agent for bleaching or disinfectanting filter media, textile, fur, paper, skin, or leather comprising a choline oxidase comprising the amino acid sequence of one of SEQ ID NO: 10 to SEQ ID NO: 15 or an amino acid sequence that is at least 83% identical to SEQ ID NO: 1, at least 72.4% identical to SEQ ID NO:2, or at least 68.9% identical to SEQ ID NO:3

2. A choline oxidase comprising the amino acid sequence of one of SEQ ID NO: 10 to SEQ ID NO: 15 or an amino acid sequence that is at least 83% identical to SEQ ID NO: 1, at least 72.4% identical to SEQ ID NO:2, or at least 68.9% identical to SEQ ID NO:3.

3. A nucleic acid encoding a choline oxidase comprising the nucleotide sequence of one of SEQ ID NO:17 to SEQ ID NO:22 or a nucleotide sequence that is at least 82% identical to SEQ ID NO:4, at least 83.3% identical to SEQ ID NO:5, or at least 81.5% identical to SEQ ID NO:6.

4. An oligonucleotide comprising the nucleotide sequence of one of SEQ ID NO:8, SEQ ID NO:9, or SEQ ID NO:23.

5. A nucleic acid probe comprising a nucleotide sequence that is at least 85% identical to SEQ ID NO:7.

6. A vector comprising the nucleic acid of claim 3.

7. A host cell that expresses a choline oxidase comprising the nucleic acid of claim 3.

8. The host cell of claim 7 wherein the host cell is a bacterium that secretes the choline oxidase from the cell.

9. The host cell of claim 8 wherein the host cell is an Escherichia coli, Bacillus licheniformis, Bacillus amyloliquefaciens, Bacillus subtilis, Bacillus alcalophilus, or Arthrobacter oxidans cell.

10. The host cell of claim 7 wherein the host cell is a eukaryotic cell that posttranslationally modifies the choline oxidase.

11. A method for producing hydrogen peroxide in situ comprising reacting a choline oxidase with a substrate in the presence of oxygen, wherein the choline oxidase belongs to the family of GMC oxidoreductases, binds FAD as a cofactor, and comprises an amino acid domain at the N-terminal region of the protein that comprises the sequence GXGXXG, wherein X denotes any amino acid.

12. The method of claim 11, wherein the GXGXXG sequence is at position 20 to 25 of the choline oxidase.

13. The method of claim 11, wherein the GXGXXG sequence is GGGSAG.

14. The method of claim 11, wherein the choline oxidase comprises the amino acid sequence of one of SEQ ID NO: 10 to SEQ ID NO: 15 or an amino acid sequence that is at least 83% identical to SEQ ID NO: 1, at least 72.4% identical to SEQ ID NO:2, or at least 68.9% identical to SEQ ID NO:3.

15. A method for bleaching, inhibiting dye transfer, or disinfecting comprising reacting the choline oxidase of claim 2 with a substrate in the presence of oxygen.

16. A detergent or bleaching agent comprising a bleaching system that produces hydrogen peroxide, and optionally comprising a synthetic surfactant, organic builder, inorganic builder, and bleaching activator, wherein the bleaching system comprises the choline oxidase of claim 1 and a substrate.

17. The detergent or bleaching agent of claim 16 wherein the substrate is choline, betaine aldehyde, or a choline derivative.

18. The detergent or bleaching agent of claim 16 wherein the choline oxidase has an activity of at least 3 U/g.

19. The detergent or bleaching agent of claim 16 wherein the detergent or bleaching agent is in the form of a flowable powder with a bulk density of 300 g/l to 1200 g/l, a pasty detergent, a liquid detergent, a non-aqueous liquid detergent, a non-aqueous paste, or an aqueous liquid detergent or paste that contains water and is packaged in an air-impermeable container from which it is released shortly before use or during the washing operation.

20. The detergent or bleaching agent of claim 16 wherein one or both of the choline oxidase and the substrate are enclosed in a substance that is impermeable to the choline oxidase and the substrate at room temperature or in the absence of water, which substance becomes permeable to the choline oxidase and the substrate under the conditions of use of the agent.

21. The detergent or bleaching agent of claim 16 further comprising
a surfactant,
a water-soluble, water-dispersible inorganic builder material,
a water-soluble organic builder substance,
a solid inorganic or organic acid or acid salt,
a heavy metal complexing agent,
a graying inhibitor,
a dye transfer inhibitor, and
a foam inhibitor.

22. A signal reagent for the production of light emission in a chemiluminescence assay, comprising the choline oxidase of claim 2, a choline oxidase substrate, and a chemiluminescence reagent.

23. The signal reagent of claim 22 wherein the choline oxidase substrate is choline, a choline derivative, or betaine aldehyde.

24. The signal reagent of claim 22 wherein the chemiluminescence reagent is luminol.


SEQUENCE LISTING (23 sequences)

SEQ ID NO: 1 - protein, 542 aa, Arthrobacter nicotinianae
  MISC_FEATURE (1)..(542): Choline oxidase from Arthrobacter nicotianae (KC2, DSMZ-ID 96-878)
TRKGEKLNIEKKDFDYIVIGGGSAGAAVASRLSEDPSVSVALVEAGPDDRGYDEVLQLDRWMELLESGLDWDYPIEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSCDFSDKPKVDPRYFTDPEGHDARVMTFGIRKAREIVAQSPMAEWAGEEQFPGKDVQTDEEIFDYLRRCHNTVYHPAGSVRMGAEDDVMSPLDPQLRVKGVSGLRVADASVMPELVTVNPNITVMMIGERCAELIQENPVEARQSELVSEA

SEQ ID NO: 2 - protein, 550 aa, Arthrobacter aurescens
  MISC_FEATURE (1)..(550): Choline oxidase from Arthrobacter aurescens (ATCC 13344)
MHVDNIENLSERGFDYIVIGGGSAGAAVAARLSEDPAVTVALVEAGPDDRNLPEILQLDRWMELLESGYDWDYPVEPQENGNSFMRHARAKVMGGCSSHNSCIAFWAPREDLDEWESKYGATGWNAAAAWPLYQRLESNEDAGPDAPHHGHDGPVHLMNVPPNDPAGVALLDACEQSGIPRAKFNDGTTVINGANFFQINRRSDGTRSSSSVSYIHPIMARENFTLLTGLRARQLVFDADKRCTGVDVVDSAFGRTHRLTAQREVILSTGAIDSPKLLMLSGIGPAEHLAQHGIEVVVDSPGVGEHLQDHPEGVVQFEAKQPMVQSSTQWWEIGIFTPTEEGLDRPDLMMHYGSVPFDMNTLRYGYPTTENGFSLTPNVTHARSRGTVRLRSRDFRDKPMVDPRYFTDPEGHDMRVMVAGIRKAREIAAQPAMAEWTGRELSPGIEAQTDEELQDYIRKTHNTVYHPVGTVRMGPVEDPMSPLDPELRVKGVSGLRVADASVMPEHVTVNPNITVMMIGERCADLIKARLGASRVEETTTTEADFSLSHA

SEQ ID NO: 3 - protein, 550 aa, Artificial
  Hybrid choline oxidase
MHVDNIENLSERGFDYIVIGGGSAGAAVAARLSEDPAVTVALVEAGPDDRNLPEILQLDRWMELLESGYDWDYPVEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSRDFRDKPMVDPRYFTDPEGHDMRVMVAGIRKAREIAAQPAMAEWTGRELSPGIEAQTDEELQDYIRKTHNTVYHPVGTVRMGPVEDPMSPLDPELRVKGVSGLRVADASVMPEHVTVNPNITVMMIGERCADLIKARLGASRVEETTTTEADFSLSHA

SEQ ID NO: 4 - DNA, 1629 bp, Arthrobacter nicotianiae
acgagaaagggcgaaaaattgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcatgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagcgacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 5 - DNA, 1650 bp, Arthrobacter aurescens
atgcacgtcgacaatattgagaacctcagcgagcgcgggttcgattacatcgtcatcggcggaggatctgccggggctgccgtcgccgcccggctgagcgaggaccccgccgttaccgttgccctggtggaagccggcccggatgaccggaacctcccggaaatcctgcaattggaccggtggatggaactgctggaatcggggtacgactgggactacccggtggaaccgcaggagaacggcaactcattcatgcgccacgcccgcgccaaggtgatgggcggctgttccagccacaattcctgcatcgctttctgggctccccgggaggacctggatgagtgggagtccaagtacggtgccaccggttggaacgctgccgcggcctggcctttgtatcagcgcttggaaagcaacgaggacgctggcccggatgccccacatcacggccatgatggtccggtgcacctgatgaacgttcctccgaatgatccggccggggttgcgctcctggacgcctgtgagcagagcggcattccccgggcaaagttcaacgacggcaccacagtgatcaacggcgctaatttcttccagatcaaccgccgttcggacggtacgcgttcctccagttctgtttcctacattcaccccatcatggctcgcgaaaacttcacgctgttgaccggcctgcgtgcccgccaactggtgttcgacgcggacaagcgttgcaccggcgtcgacgtcgttgactctgccttcggcaggacacacaggctcaccgcgcagcgcgaggtcatcctctccacgggcgccattgattcccccaagctgctcatgctttccggaatcggcccggccgagcacctggctcaacatggcatcgaggtggtggtggactcccccggtgttggcgagcatctgcaggaccacccggagggcgtggtgcagttcgaggccaagcagcccatggttcagtcgtccacccaatggtgggagatcggcatcttcactcccaccgaggagggactggaccgcccggacctgatgatgcactacggctccgtgccgttcgacatgaacaccctccgctacggttaccccaccacggagaacggcttcagccttaccccgaacgtcacgcacgcccgttcccgcgggaccgtccgtttgcgcagcagggacttccgcgacaagcccatggtggacccgcggtacttcaccgatcccgaggggcacgacatgcgcgtcatggtggccggcatccgcaaggcccgggaaatcgcggctcagccggccatggccgagtggaccggacgggagctctcccccggaatcgaggcccagactgatgaggagcttcaggactacatccgcaaaacccacaacaccgtctaccaccccgtcggtaccgtccgcatgggcccggtggaggaccccatgtctcccttggatccggagctgcgtgtcaaaggcgtcagcggcctacgggtggcagacgcctccgtgatgcccgaacacgtcacagtgaaccccaacatcaccgtgatgatgatcggcgagcgctgtgctgatctcatcaaagcccggctgggtgccagccgggtggaggaaaccacgacgacggaggcggacttcagcttgtcccacgcc

SEQ ID NO: 6 - DNA, 1650 bp, Artificial
  Hybrid choline oxidase
atgcacgtcgacaatattgagaacctcagcgagcgcgggttcgattacatcgtcatcggcggaggatctgccggggctgccgtcgccgcccggctgagcgaggaccccgccgttaccgttgccctggtggaagccggcccggatgaccggaacctcccggaaatcctgcaattggaccggtggatggaactgctggaatcggggtacgactgggactacccggtggaaccgcaggagaacggcaactcattcatgcgccacgcccgcgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagcagggacttccgcgacaagcccatggtggacccgcggtacttcaccgatcccgaggggcacgacatgcgcgtcatggtggccggcatccgcaaggcccgggaaatcgcggctcagccggccatggccgagtggaccggacgggagctctcccccggaatcgaggcccagactgatgaggagcttcaggactacatccgcaaaacccacaacaccgtctaccaccccgtcggtaccgtccgcatgggcccggtggaggaccccatgtctcccttggatccggagctgcgtgtcaaaggcgtcagcggcctacgggtggcagacgcctccgtgatgcccgaacacgtcacagtgaaccccaacatcaccgtgatgatgatcggcgagcgctgtgctgatctcatcaaagcccggctgggtgccagccgggtggaggaaaccacgacgacggaggcggacttcagcttgtcccacgcc

SEQ ID NO: 7 - DNA, 902 bp, Artificial
  Probe for isolating a choline oxidase
caaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgc

SEQ ID NO: 8 - DNA, 34 bp, Artificial
  Primer for cloning a choline oxidase
catgccatggcaaagggcgaaaaattgaacattg

SEQ ID NO: 9 - DNA, 31 bp, Artificial
  Primer for cloning a choline oxidase
cccaagcttctaggcttcgctaaccagttcg

SEQ ID NO: 10 - protein, 542 aa, Artificial
  Expression product of nucleic acid sequence KC2 in E. coli
MAKGEKLNIEKKDFDYIVIGGGSAGAAVASRLSEDPSVSVALVEAGPDDRGYDEVLQLDRWMELLESGLDWDYPIEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSCDFSDKPKVDPRYFTDPEGHDARVMTFGIRKAREIVAQSPMAEWAGEEQFPGKDVQTDEEIFDYLRRCHNTVYHPAGSVRMGAEDDVMSPLDPQLRVKGVSGLRVADASVMPELVTVNPNITVMMIGERCAELIQENPVEARQSELVSEA

SEQ ID NO: 11 - protein, 542 aa, Artificial
  Expression product of nucleic acid sequence KC2 M85V
MAKGEKLNIEKKDFDYIVIGGGSAGAAVASRLSEDPSVSVALVEAGPDDRGYDEVLQLDRWMELLESGLDWDYPIEPQENGNSFVRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSCDFRDKPKVDPRYFTDPEGHDARVMTFGIRKAREIVAQSPMAEWAGEEQFPGKDVQTDEEIFDYLRRCHNTVYHPAGSVRMGAEDDVMSPLDPQLRVKGVSGLRVADASVMPELVTVNPNITVMMIGERCAELIQENPVEARQSELVSEA

SEQ ID NO: 12 - protein, 542 aa, Artificial
  Expression product of nucleic acid sequence KC2 A27V
MAKGEKLNIEKKDFDYIVIGGGSAGAVVASRLSEDPSVSVALVEAGPDDRGYDEVLQLDRWMELLESGLDWDYPIEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSCDFSDKPKVDPRYFTDPEGHDARVMTFGIRKAREIVAQSPMAEWAGEEQFPGKDVQTDEEIFDYLRRCHNTVYHPAGSVRMGAEDDVMSPLDPQLRVKGVSGLRVADASVMPELVTVNPNITVMMIGERCAELIQENPVEARQSELVSEA

SEQ ID NO: 13 - protein, 542 aa, Artificial; identical to SEQ ID NO: 11
  Expression product of nucleic acid sequence KC2 M85V2

SEQ ID NO: 14 - protein, 536 aa, Artificial
  Expression product of nucleic acid sequence KC2s
MNIEKKDFDYIVIGGGSAGAAVASRLSEDPSVSVALVEAGPDDRGYDEVLQLDRWMELLESGLDWDYPIEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSCDFSDKPKVDPRYFTDPEGHDARVMTFGIRKAREIVAQSPMAEWAGEEQFPGKDVQTDEEIFDYLRRCHNTVYHPAGSVRMGAEDDVMSPLDPQLRVKGVSGLRVADASVMPELVTVNPNITVMMIGERCAELIQENPVEARQSELVSEA

SEQ ID NO: 15 - protein, 536 aa, Artificial; identical to SEQ ID NO: 14
  Expression product of nucleic acid sequence KC2s in E. coli

SEQ ID NO: 16 - protein, 547 aa, Arthrobacter globiformis
MHIDNIENLSDREFDYIVVGGGSAGAAVAARLSEDPAVSVALVEAGPDDRGVPEVLQLDRWMELLESGYDWDYPIEPQENGNSFMRHARAKVMGGCSSHNSCIAFWAPREDLDEWEAKYGATGWNAEAAWPLYKRLETNEDAGPDAPHHGDSGPVHLMNVPPKDPTGVALLDACEQAGIPRAKFNTGTTVVNGANFFQINRRADGTRSSSSVSYIHPIVEQENFTLLTGLRARQLVFDADRRCTGVDIVDSAFGRTHRLTARNEVVLSTGAIDTPKLLMLSGIGPAAHLAEHGIEVLGGLPRRGRAPAGPPGRRGAVRGQAAHGRRVHAVVGDRHLHPHRGRPGPPRPDDALRLRAVRHEHPAARLPHHGERASASPRTSRTPAPAALSGCAAATSAISPWSTRATSPTQKGHDMRVMVAGIRKAREIAAQPAMAEWTGRELSPGVEAQTDEELQDYIRKTHNTVYHPVGTVRMGAVEDEMSPLDPELRVKGVTGLRVGDASVMPEHVTVNPNITVMMIGERCADLIRSARAGETTTADAELSAALA

SEQ ID NO: 17 - DNA, 1629 bp, Artificial
  Choline oxidase from A. nicotianiae (KC 2, DSMZ-ID 96-878) cloned in E. coli
atggcaaagggcgaaaaattgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcatgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagcgacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 18 - DNA, 1629 bp, Artificial
  KC2 M85V, mutant of KC2 in E. coli
atggcaaagggcgaaaaattgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcgtgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagagacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 19 - DNA, 1629 bp, Artificial
  KC2 A27V, mutant of KC2 in E. coli
atggcaaagggcgaaaaattgaacattgaaaagaaggacttcgactacattgtgatcggcggcggttctgccggcgctgtggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcatgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagcgacaagccgaaggtcgaccctcgctacttcaccgatccagagggccacgacgcccgggtcatgactttcggcatccgcaaggcccgcgagatcgtcgcccagtcgccgatggcagagtgggctggcgaggagcagttcccgggcaaggacgtgcagactgacgaagagatcttcgactacttgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 20 - DNA, 1629 bp, Artificial
  KC2 M85V2, mutant of KC2 M85V
atggcaaagggcgaaaaattgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcgtgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggatggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagagacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 21 - DNA, 1611 bp, Artificial
  KC2s (original form derived from KC2)
ttgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcatgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagcgacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 22 - DNA, 1611 bp, Artificial
  KC2s (cloned in E.coli)
atgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcatgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagcgacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 23 - DNA, 40 bp, Artificial
  KC2spETNF - primer for the production of KC2s
ttccatatgaacattgaaaagaaggacttcgactacattg